SEQUENCE LISTING (22 sequences)

SEQ ID NO: 1 - DNA, 649 bp, Nucleotide Sequence of VEGF165
  CDS (17)..(589)
tcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagcgtttcgggaaccagatctctcaccagg

SEQ ID NO: 2 - protein, 191 aa, Nucleotide Sequence of VEGF165
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 3 - DNA, 1094 bp, Nucleotide Sequence of SOM175
  CDS (3)..(623)
ccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaaggggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaa

SEQ ID NO: 4 - protein, 207 aa, Nucleotide Sequence of SOM175
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 5 - DNA, 993 bp, Nuc. Seq. of SOM175 Absent Exon 6
  CDS (3)..(566)
ccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagatagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaaggggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaa

SEQ ID NO: 6 - protein, 188 aa, Nuc. Seq. of SOM175 Absent Exon 6
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 7 - DNA, 858 bp, Nuc. Seq. of SOM175 Absent Exons 6&7
  CDS (3)..(431)
ccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagataggtgccggaagctgcgaaggtgacacatggcttttcagactcagcagggtgacttgcctcagaggctatatcccagtgggggaacaaaggggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaa

SEQ ID NO: 8 - protein, 143 aa, Nuc. Seq. of SOM175 Absent Exons 6&7
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRCRKLRR

SEQ ID NO: 9 - DNA, 910 bp, Nuc. Seq. of SOM175 Absent Exon 4
  CDS (3)..(305)
ccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtggggaacaaagaggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcc

SEQ ID NO: 10 - protein, 101 aa, Nuc. Seq. of SOM175 Absent Exon 4
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQT

SEQ ID NO: 11 - DNA, 42 bp, Oligonucleotide
accaccacctccctgggctggcatgtggcacgtgcataaacg

SEQ ID NO: 12 - DNA, 42 bp, Oligonucleotide
agttgtttgaccacattgcccatgagttccatgctcagaggc

SEQ ID NO: 13 - DNA, 38 bp, Oligonucleotide
gatcctggggctggagtgggatggatgatgtcagctgg

SEQ ID NO: 14 - DNA, 40 bp, Oligonucleotide
gcgggcagaggatcctggggctgtctggcctcacagcact

SEQ ID NO: 15 - DNA, 236 bp, Human SOM175
atgaggggccaggtacgtgaggtctcccacaggcccctggaaagaatacttacatctgctcccatggtgtatgcaggtccgagatgctgaatacagatcctcatgcaggtgtcaggcaacttttcaagacctaaagacaggtgagtctttctcctccgtaggctgcctccagccccaggccccccactccagccccagacccagacacctgtagccctgctcaggtgccgaggtga

SEQ ID NO: 16 - DNA, 1242 bp, mVRF
  CDS (166)..(789)
gcacgagctcaggccgtcgctgcggcgctgcgttgcgctgcctgcgcccagggctcgggagggggccgcggaggagccgccccctgcgccccgccccgggtccccgggtccgcgccatggggcggctctggctgacccccccccacaccgccgggctagggcccgatgagccccctgctgcgtcgcctgctgcttgttgcactgctgcagctggctcgcacccaggcccctgtgtcccagtttgatggccccagtcaccagaagaaagtggtgccatggatagacgtttatgcacgtgccacatgccagcccagggaggtggtggtgcctctgagcatggaactcatgggcaatgtggtcaaacaactagtgcccagctgtgtgactgtgcagcgctgtggtggctgctgccctgacgatggcctggaatgtgtgcccactgggcaacaccaagtccgaatgcagatcctcatgatccagtacccgagcagtcagctgggggagatgtccctgggagaacacagccaatgtgaatgcagacctaaaaaaaaggagagtgctgtgaggccagacagggttgccataccccaccaccgtccccagccccgctctgttccgggctgggactctaccccgggagcaccctccccagctgacatcatccatcccactccagccccaggatcctctgcccgccttgcacccagcgccgccaacgccctgacccccggacctgccgttgccgctgtagacgccgccgcttcctccattgccaagggcggggcttagagctcaacccagacacctgtaggtgccggaagccgcgaaagtgacaagctgctttccagactccacgggcccggctgcttttatggccctgcttcacagggagaagagtggagcacaggcgtaacctcctcagtctgggaggtcactgccccaggacctggaccttttagagagctctctcgccatcttttatctcccagagctgccatctaacaattgtcaaggaacctcatgtctcacctcaggggccagggtactctctcacttaaccaccctggtcaagtgagcatcttctggctggctgtctcccctcactatgaaaaccccaaacttctaccaataacgggatttgggttctgttatgataactgtgacacacacacacactcacactctgataaaagagatggagacactaaaaaaaaaaaaaaaaaaaaaaaaaaaaaaaaaaaa

SEQ ID NO: 17 - protein, 207 aa, mVRF
MSPLLRRLLLVALLQLARTQAPVSQFDGPSHQKKVVPWIDVYARATCQPREVVVPLSMELMGNVVKQLVPSCVTVQRCGGCCPDDGLECVPTGQHQVRMQILMIQYPSSQLGEMSLGEHSQCECRPKKKESAVRPDRVAIPHHRPQPRSVPGWDSTPGAPSPADIIHPTPAPGSSARLAPSAANALTPGPAVAAVDAAASSIAKGGA

SEQ ID NO: 18 - protein, 188 aa, mVRF167
MSPLLRRLLLVALLQLARTQAPVSQFDGPSHQKKVVPWIDVYARATCQPREVVVPLSMELMGNVVKQLVPSCVTVQRCGGCCPDDGLECVPTGQHQVRMQILMIQYPSSQLGEMSLGEHSQCECRPKKKESAVRPDSPRILCPPCTQRRQRPDPRTCRCRCRRRRFLHCQGRGLELNPDTCRCRKPRK

SEQ ID NO: 19 - protein, 188 aa, hVRF167; identical to SEQ ID NO: 6

SEQ ID NO: 20 - protein, 71 aa, mVRF186
RVAIPHHRPQPRSVPGWDSTPGAPSPADIIHPTPAPGSSARLAPSAANALTPGPAVAAVDAAASSIAKGGA

SEQ ID NO: 21 - protein, 71 aa, hVRF186
RAATPHHRPQPRSVPGWDSAPGAPSPADITHPTPAPGPSAHAAPSTTSALTPGPAAAAADAAASSVAKGGA

SEQ ID NO: 22 - protein, 214 aa, mVEGF188
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPEKKSVRGKGKGQKRKRKKSRFKSWSVHCEPCSERRKHLFVQDPQTCKCSCKNTDSRCKARQLELNERTCRCDKPRR

(19) United States
(12) Patent Application Publication
(10) Pub. No.: US 2007/0135344 A1
(43) Pub. Date: Jun. 14, 2007

(54) Novel growth factor and a genetic sequence encoding same

(76) Inventors: Nicholas Kim Hayward, Grange (AU); Gunther Weber, Stockholm (SE); Sean Grimmond, The Gap (AU); Magnus Nordenskjold, Stockholm (SE); Catharina Larsson, Stockholm (SE)

Correspondence Address: CROWELL & MORING LLP;INTELLECTUAL PROPERTY GROUP, P.O. BOX 14300, WASHINGTON, DC, 20044-4300, US

(21) Appl. No.: 11/602,055

(22) Filed: Nov. 20, 2006

Related U.S. Application Data
(63) Continuation of application No. 08/765,588, filed on Apr. 25, 1997, now abandoned.

(30) Foreign Application Priority Data
Mar. 2, 1995 (AU) PN 1457/95
Nov. 20, 1995 (AU) PN 6647/95
Dec. 22, 1995 (AU) PN 7274/95

(51) Int. Cl. A61K 38/18; C07K 14/475; C07K 16/22; C07H 21/04; C12P 21/06

(52) U.S. Cl. 514/12; 435/69.1; 435/320.1; 435/325; 530/388.22; 530/388.25; 536/23.5

(57) ABSTRACT

The present invention relates to an isolated molecule having vascular endothelial growth factor-like properties and to a genetic sequence encoding the molecule.

The present invention relates generally to an isolated molecule having vascular endothelial growth factor-like properties and to a genetic sequence encoding same. The molecule will be useful in the development of a range of therapeutics and diagnostics useful in the treatment, prophylaxis and/or diagnosis of conditions requiring enhanced or diminished vasculature and/or vascular permeability. The molecule of the present invention is also a useful effector of primary and central neurons and is capable of inducing astroglial proliferation.

Bibliographic details of the publications referred to by author in this specification are collected at the end of the description. Sequence Identity Numbers (SEQ ID NOs.) for the nucleotide and amino acid sequences referred to in the specification are defined following the bibliography.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Vascular endothelial growth factor (hereinafter referred to as “VEGF”), also known as vasoactive permeability factor, is a secreted, covalently linked homodimeric glycoprotein that specifically activates endothelial tissues (Senger et al., 1993). A range of functions have been attributed to VEGF such as its involvement in normal angiogensis including formation of the corpus luteun (Yan et al., 1993) and placental development (Sharkey et al., 1993), regulation of vascular permeability (Senger et al., 1993), inflammatory angiogenesis (Sunderkotter et al., 1994) and autotransplantation (Dissen et al., 1994) and human diseases such as tumour promoting angiogenesis (Folkman & Shing, 1992), rheumatoid arthritis (Koch et al., 1994) and diabetes related retinopathy (Folkman & Shing, 1992).

VEGF is, therefore, an important molecule making it a potentially valuable target for research into therapeutics, prophylactics and diagnostic agents based on VEGF or its activities. There is also a need to identify homologues or otherwise related molecules for use as an alternative to VEGF or in conjunction with VEGF.

In work leading up to the present invention, the inventors sought the multiple endocrine neoplasia type I susceptibility gene (MEN1). Surprisingly, the inventors discovered that a genetic sequence excluded as a candidate for the MEN1 gene was nevertheless a new growth factor having some similarity to VEGF. Furthermore, the growth factor of the present invention is an effector molecule for primary and central neurons.

Accordingly, one aspect of the present invention comprises a biologically isolated proteinaceous molecule comprising a sequence of amino acids which:
(i) is at least about 15% similar to the amino acid sequence set forth in SEQ ID NO:2; and
(ii) is at least 5% dissimilar to the amino acid sequence set forth in SEQ ID NO:2.

Another aspect of the present invention provides a biologically isolated proteinaceous molecule having the following characteristics:
(i) comprises an amino acid sequence having at least about 15% similarity but at least about 5% dissimilarity to all or part of the amino acid sequence set forth in SEQ ID NO:2;
(ii) exhibits at least one property in common with VEGF.

A related aspect of the present invention contemplates a biologically isolated proteinaceous molecule having the following characteristics:
(i) comprises an amino acid sequence having at least about 15% similarity but at least about 5% dissimilarity to the amino acid sequence set forth in SEQ ID NO:2;
(ii) exhibits at least one of the following properties:
   (a) ability to induce proliferation of vascular endothelial cells;
   (b) ability to interact with flt-1/flk-1 family of receptors;
   (c) ability to induce cell migration, cell survival and/or an increase in intracellular levels of alkaline phosphatase.

By “biologically isolated” is meant that the molecule has undergone at least one step of purification from a biological source. Preferably, the molecule is also biologically pure meaning that a composition comprises at least about 20%, more preferably at least about 40%, still more preferably at least about 65%, even still more preferably at least about 80-90% or greater of the molecule as determined by weight, activity or other convenient means, relative to other compounds in the composition. Most preferably, the molecule is sequencably pure.

Another preferred aspect of the present invention provides the molecule in recombinant form.

According to this aspect of the present invention, there is provided a recombinant molecule comprising a sequence of amino acids which:
(i) is at least about 15% similar to the amino acid sequence set forth in SEQ ID NO:2; and
(ii) is at least 5% dissimilar to the amino acid sequence set forth in SEQ ID NO:2.

A related aspect of the present invention is directed to a recombinant molecule having the following characteristics:
(i) comprises an amino acid sequence having at least about 15% similarity but at least about 5% dissimilarity to all or part of the amino acid sequence set forth in SEQ ID NO:2;
(ii) exhibits at least one property in common with VEGF.

A further related aspect of the present invention contemplates a recombinant molecule having the following characteristics:
(i) comprises an amino acid sequence having at least about 15% similarity but at least about 5% dissimilarity to the amino acid sequence set forth in SEQ ID NO:2;
(ii) exhibits at least one of the following properties:
   (a) ability to induce proliferation of vascular endothelial cells;
   (b) ability to interact with flt-1/flk-1 family of receptors;
   (c) ability to induce cell migration, cell survival and/or an increase in intracellular levels of alkaline phosphatase.

The present invention also contemplates genomic or partial genome clones encoding a proteinaceous molecule having at least about 15% amino acid similarity but at least about 5% dissimilarity to SEQ ID NO:1.

The amino acid sequence set forth in SEQ ID NO:2 corresponds to human VEGF (referred to herein as “VEGF₁₆₅”). Accordingly, the molecule of the present invention is VEGF-like or is a homologue of VEGF but comprises an amino acid sequence which is similar but non-identical to the amino sequence of VEGF. Although the present invention is exemplified using a human VEGF-like molecule, this is done with the understanding that the instant invention contemplates the homologous molecule and encoding sequence from other mammals such as livestock animals (e.g. sheep, pigs, horses and cows), companion animals (e.g. dogs and cats) and laboratory test animals (e.g. mice, rats, rabbits and guinea pigs) as well as non-mammals such as birds (e.g. poultry birds), fish and reptiles. In a most preferred embodiment, the VEGF-like molecule is of human origin and encoded by a gene located at chromosome 11q13. The present invention extends, therefore, to the genomic sequence or part thereof encoding the subject VEGF-like molecule.

Preferably, the percentage similarity is at least about 30%, more preferably at least about 40%, still more preferably at least about 50%, still even more preferably at least about 60-70%, yet even more preferably at least about 80-95% to all or part of the amino acid sequence set forth in SEQ ID NO:2.

In a particularly preferred embodiment, the VEGF-like molecule of the present invention comprises a sequence of amino acids as set forth in SEQ ID NO:4 or is a part, fragment, derivative or analogue thereof. Particularly preferred similarities include about 19-20%, and 29-30%. Reference herein to derivatives also includes splice variants. Accordingly, the present invention extends to splice variants of SOM175. Examples of splice variants contemplated by the present invention include but are not limited to variants with an amino acid sequence substantially as set forth in at least one of SEQ ID NO:6, SEQ ID NO:8 and/or SEQ ID NO:10 or mutants or derivatives or further splice variants thereof.

Another embodiment provides a recombinant molecule having the following characteristics:
(i) an amino acid sequence substantially as set forth in SEQ ID NO:4 or having at least about 15% similarity to all or part thereof provided that said amino acid sequence is at least about 5% dissimilar to all or part of the amino acid sequence set forth in SEQ ID NO:2;
(ii) exhibits at least one biological property in common with VEGF.

Another embodiment provides a recombinant molecule having the following characteristics:
(i) an amino acid sequence substantially as set forth in SEQ ID NO:6 or having at least about 15% similarity to all or part thereof provided that said amino acid sequence is at least about 5% dissimilar to all or part of the amino acid sequence set forth in SEQ ID NO:2;
(ii) exhibits at least one biological property in common with VEGF.

Another embodiment provides a recombinant molecule having the following characteristics:
(i) an amino acid sequence substantially as set forth in SEQ ID NO:8 or having at least about 15% similarity to all or part thereof provided that said amino acid sequence is at least about 5% dissimilar to all or part of the amino acid sequence set forth in SEQ ID NO:2;
(ii) exhibits at least one biological property in common with VEGF.

Another embodiment provides a recombinant molecule having the following characteristics:
(i) an amino acid sequence substantially as set forth in SEQ ID NO:10 or having at least about 15% similarity to all or part thereof provided that said amino acid sequence is at least about 5% dissimilar to all or part of the amino acid sequence set forth in SEQ ID NO:2;
(ii) exhibits at least one biological property in common with VEGF.

Such properties of VEGF include at least one of:
(a) ability to induce proliferation of vascular endothelial cells;
(b) an ability to interact with flt-1/flk-1 family of receptors;
(c) an ability to induce cell migration, cell survival and/or an increase in intracellular levels of alkaline phosphatase.

In accordance with the present invention, a preferred similarity is at least about 40%, more preferably at least about 50% and even more preferably at least about 65% similarity.

Still a further aspect of the present invention contemplates a peptide fragment corresponding to a portion of the amino acid sequence set forth in SEQ ID NO:4 or a splice variant thereof such as set forth in SEQ ID NO:6, SEQ ID NO:8 or SEQ ID NO:10 or a chemical equivalent thereof. The biologically isolated or recombinant molecule of the present invention may be naturally glycosylated or may comprise an altered glycosylation pattern depending on the cells from which it is isolated or synthesised. For example, if produced by recombinant means in prokaryotic organisms, the molecule would be non-glycosylated. The molecule may be a full length, naturally occurring form or may be a truncated or otherwise derivatised form.

Yet another aspect of the present invention is directed to a nucleic acid molecule encoding the VEGF-like molecule herein described. More particularly, the present invention provides a nucleic acid molecule comprising a sequence of nucleotides substantially as set forth in SEQ ID NO:3 or having at least 15% similarity to all or part thereof or being capable of hybridising under low stringency conditions to a reverse complement of the nucleotide sequence as set forth in SEQ ID NO:3 provided that the nucleic acid sequence having at least 15% similarity but at least 30% dissimilarity to the nucleotide sequence as set forth in SEQ ID NO:3. The nucleotide sequence set forth in SEQ ID NO:3 is also referred to herein as “SOM175”. Preferably, the percentage dissimilarity is about 35%, more preferably about 39% and even more preferably about 40-50% or greater.

For the purposes of defining the level of stringency, reference can conveniently be made to Sambrook et al (1989) at pages 9.47-9.51 which is herein incorporated by reference where the washing steps disclosed are considered high stringency. A low stringency is defined herein as being in 4-6×SSC/0.1-0.5% w/v SDS at 37-45° C. for 2-3 hours. Depending on the source and concentration of nucleic acid involved in the hybridisation, alternative conditions of stringency may be employed such as medium stringent conditions which are considered herein to be 14×SSC/0.25-0.5% w/v SDS at ≧45° C. for 2-3 hours or high stringent conditions considered herein to be 0.1-1×SSC/0.1% w/v SDS at 60° C. for 1-3 hours.

The present invention further contemplates a nucleic acid molecule which encodes a VEGF-like molecule as hereinbefore described having at least 15% nucleotide sequence homology to SEQ ID NO:3. Preferred levels of homology include at least about 40%, more preferably around 60-70%.

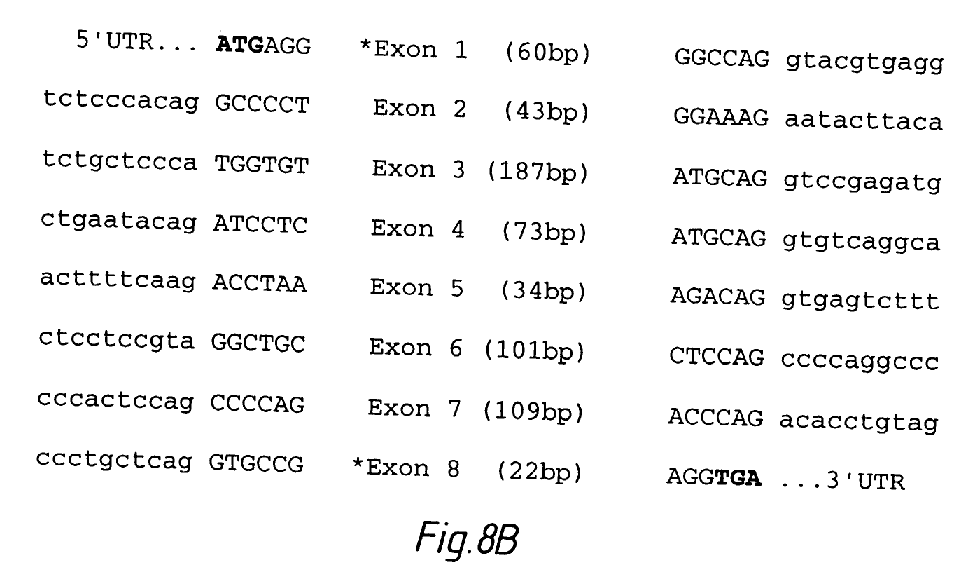

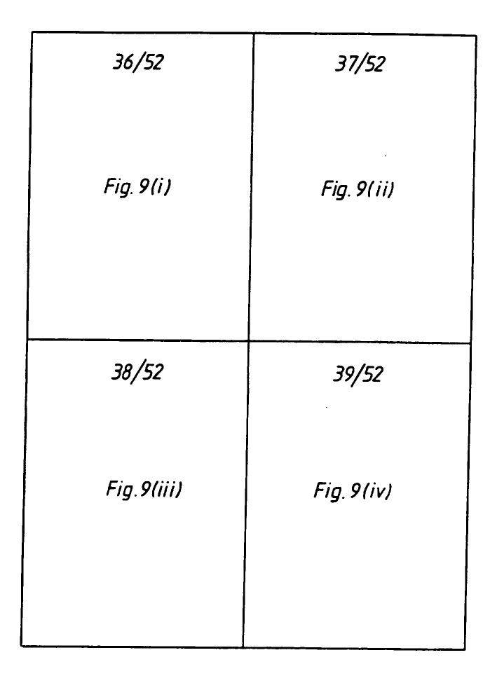

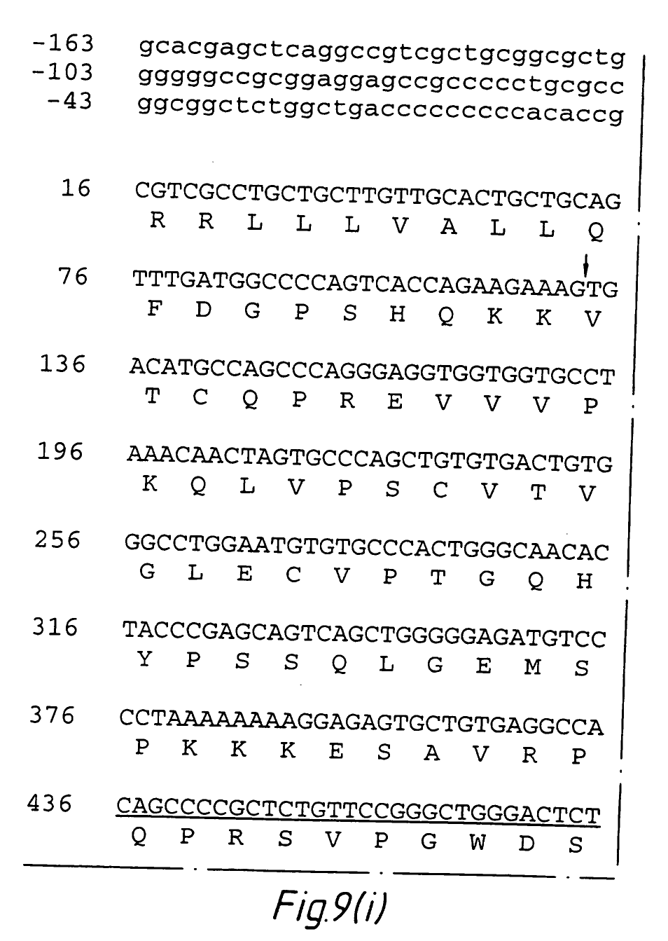

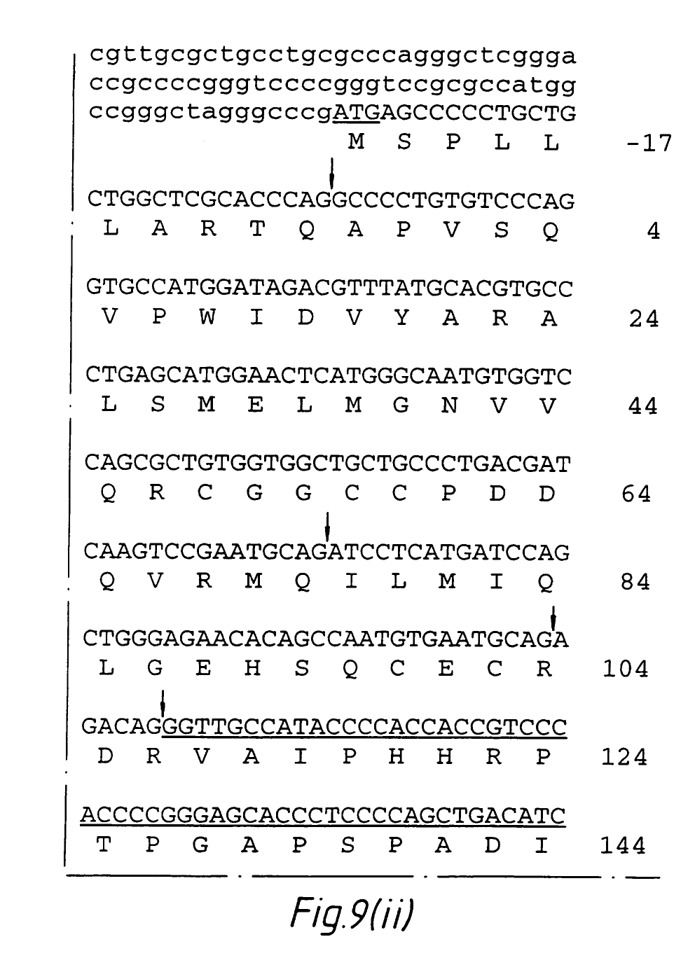

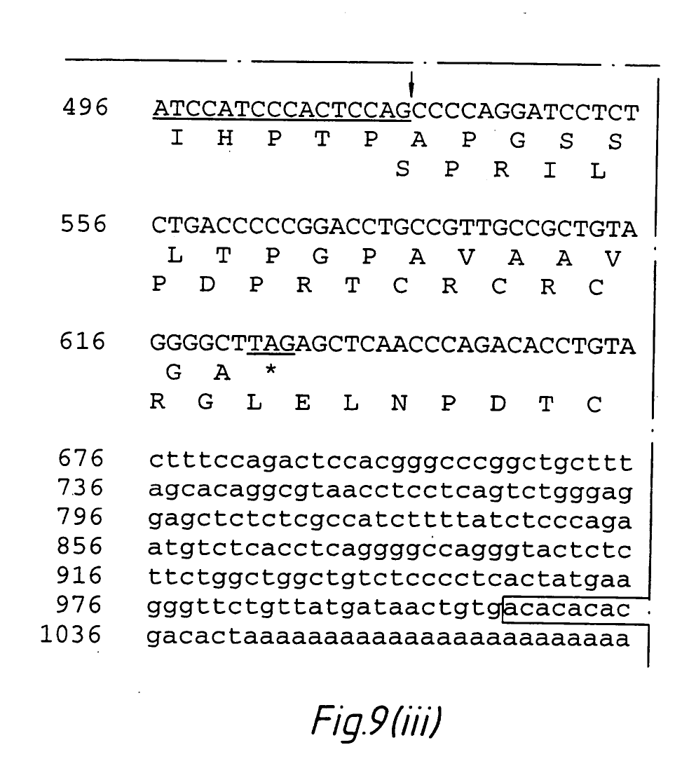

The present invention is further directed to the murine homologue of human VEGF (referred to herein as “mVRF”). The mVRF has approximately 85% identity and 92% conservation of amino acid residues over the entire coding region compared to human VEGF. The mVRF is encoded by a nucleic acid molecule comprising a nucleotide sequence substantially as set forth in FIG. 9.

The VEGF-like molecule of the present invention will be useful in the development of a range of therapeutic and/or diagnostic applications alone or in combination with other molecules such as VEGF. The present invention extends, therefore, to pharmaceutical compositions comprising the VEGF-like molecule or parts, fragments, derivatives, homologues or analogues thereof together with one or more pharmaceutically acceptable carriers and/or diluents. Furthermore, the present invention extends to vectors comprising the nucleic acid sequence set forth in SEQ ID NO:3 or having at least about 15%, more preferably about 40% and even more preferably around 60-70% similarity thereto but at least 30% and more preferably around 39% dissimilarity thereto and host cells comprising same. In addition, the present invention extends to ribozymes and antisense molecules based on SEQ ID NO:3 as well as neutralizing antibodies to the VEGF-like molecule. Such molecules may be useful in ameliorating the effects of, for example, over expression of VEGF-like genes leading to angiogenesis or vascularization of tumours.

Another aspect of the present invention contemplates a method of inducing astroglial proliferation in a mammal, said method comprising administering to said mammal an effective amount of a recombinant proteinaceous molecule having the characteristics:
(i) comprises an amino acid sequence having at least about 15% similarity but at least about 5% dissimilarity to the sequence set forth in SEQ ID NO:2;
(ii) exhibits at least one property in common with vascular endothelial growth factor (VEGF),
   said administration being for a time and under conditions sufficient to induce astroglial proliferation.

Preferably, the recombinant proteinaceous molecule comprises the amino acid sequence set forth in SEQ ID NO:3 or SEQ ID NO:6.

A further aspect of the present invention provides a method of promoting neural survival and/or proliferation in a mammal, said method comprising administering to said mammal an effective amount of a recombinant proteinaceous molecule having the characteristics:
(i) comprises an amino acid sequence having at least about 15% similarity but at least about 5% dissimilarity to the sequence set forth in SEQ ID NO:2;
(ii) exhibits at least one property in common with vascular endothelial growth factor (VEGF),
   said administration being for a time and under conditions sufficient to induce astroglial proliferation.

Preferably, the recombinant proteinaceous molecule comprises the amino acid sequence set forth in SEQ ID NO:3 or SEQ ID NO:6.

The present invention also contemplates antibodies to the VEGF-like molecule or nucleic acid probes to a gene encoding the VEGF-like molecule which are useful as diagnostic agents.

The present invention is further described by reference to the following non-limiting Figures and/or Examples.

In the Figures:

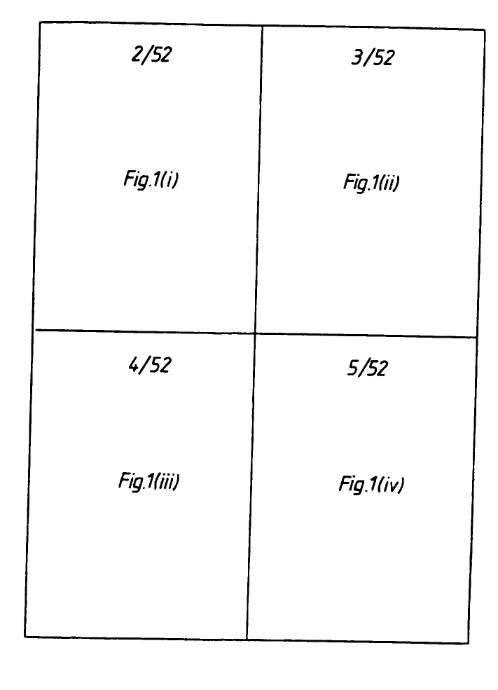

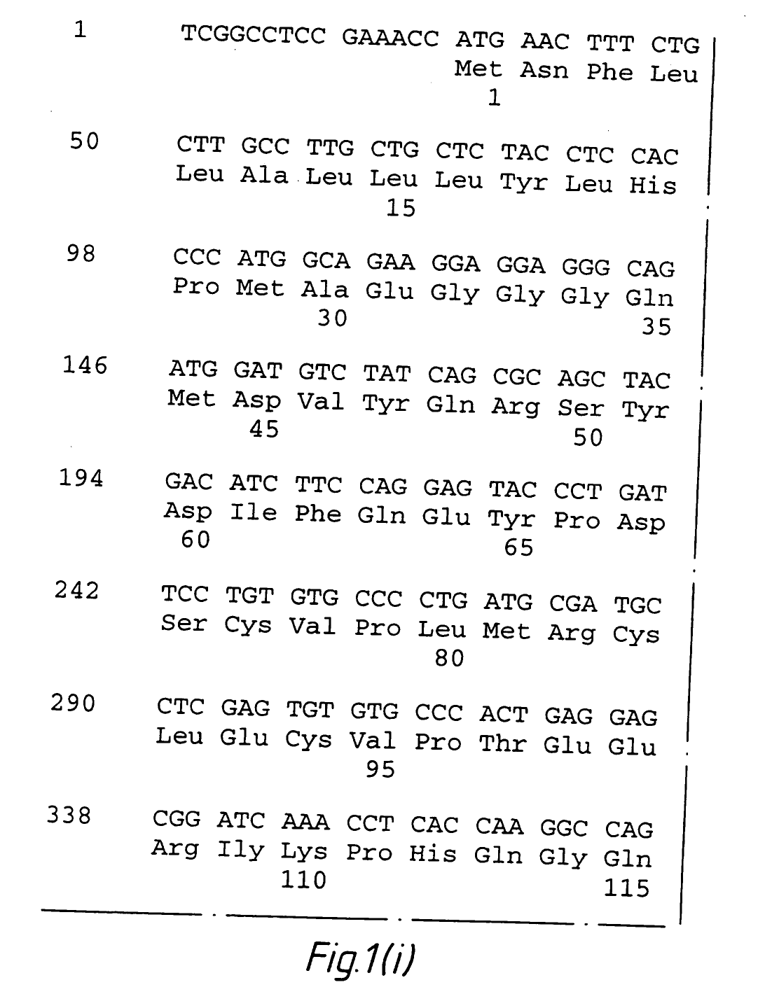

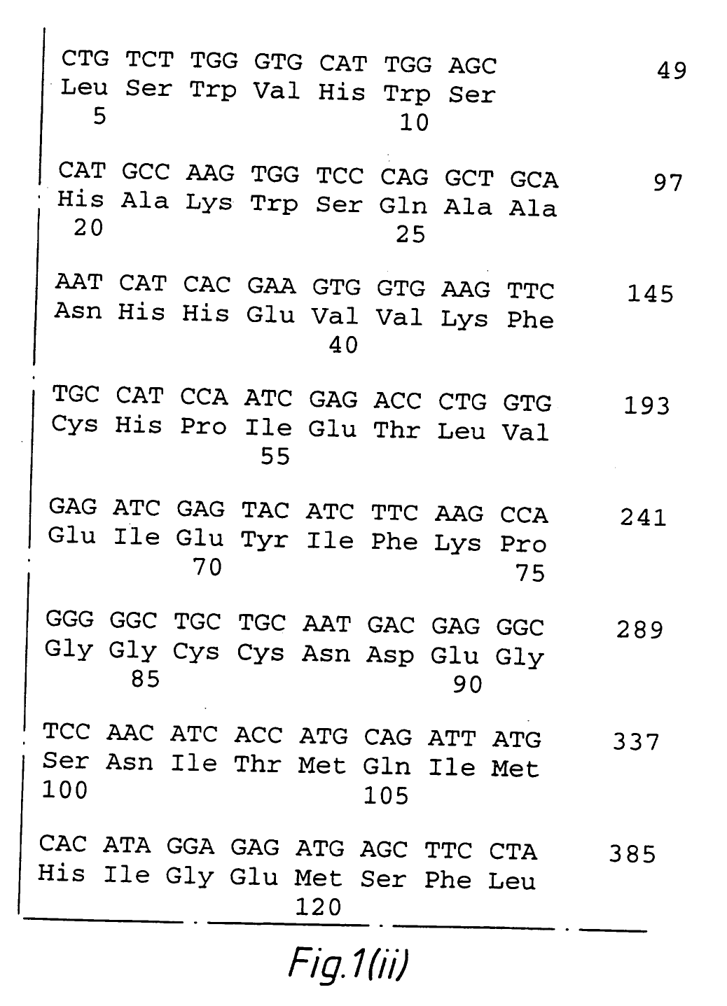

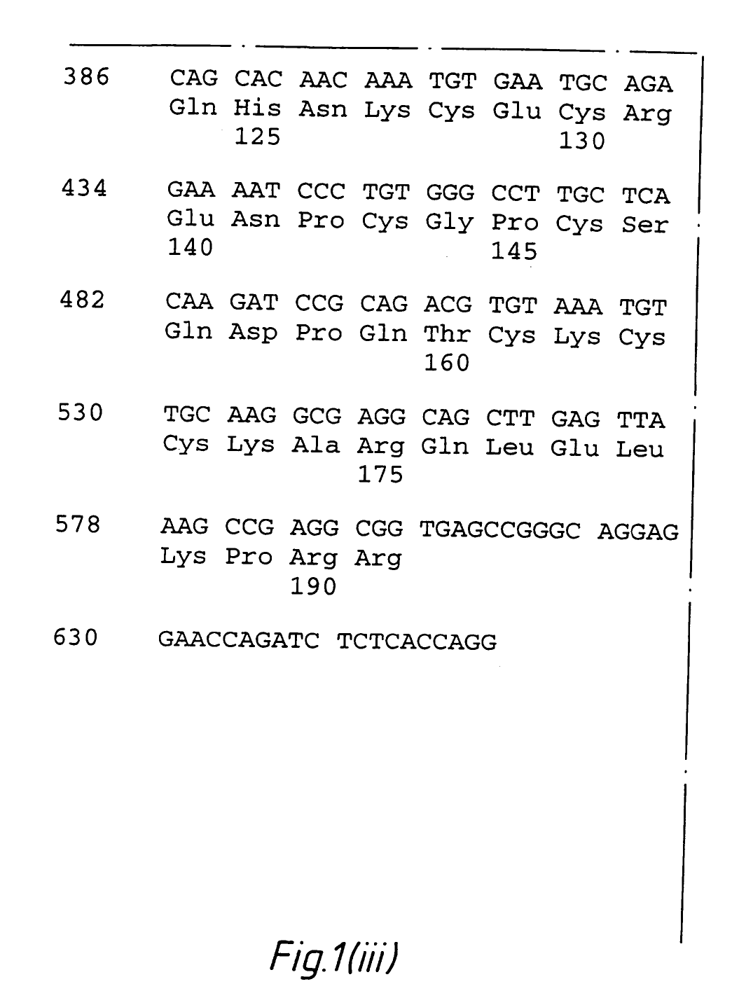

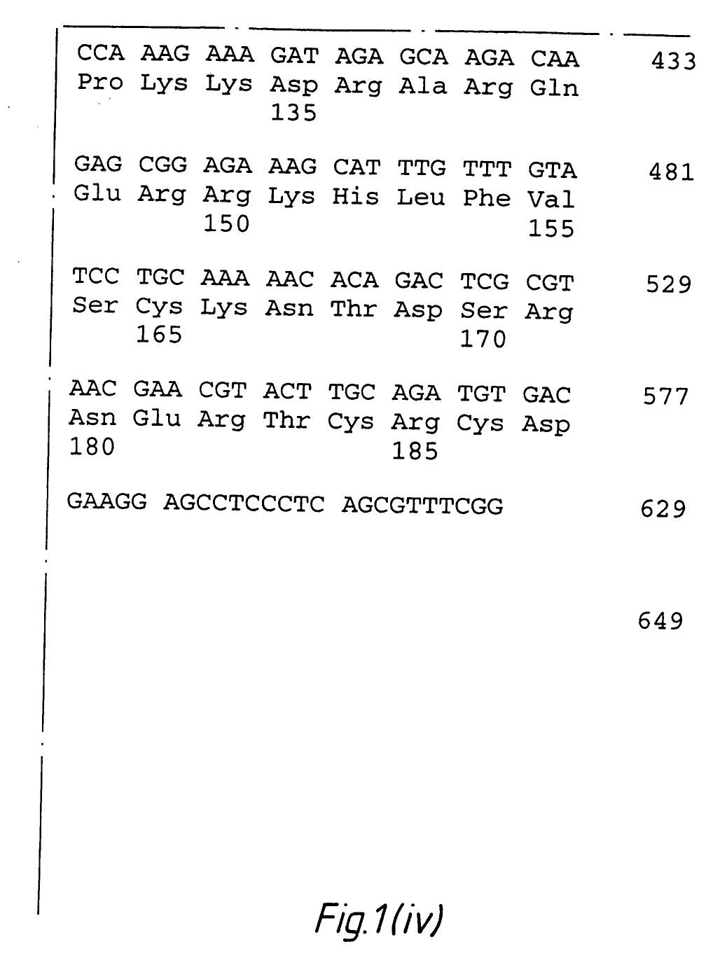

FIG. 1 Nucleotide sequence [SEQ ID NO:1] and corresponding amino acid sequence [SEQ ID NO:2] of VEGF₁₆₅.

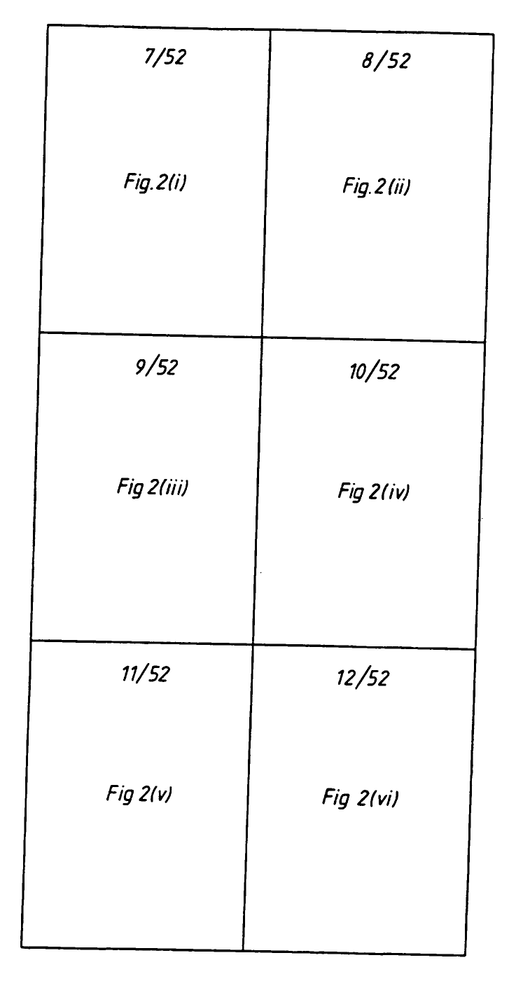

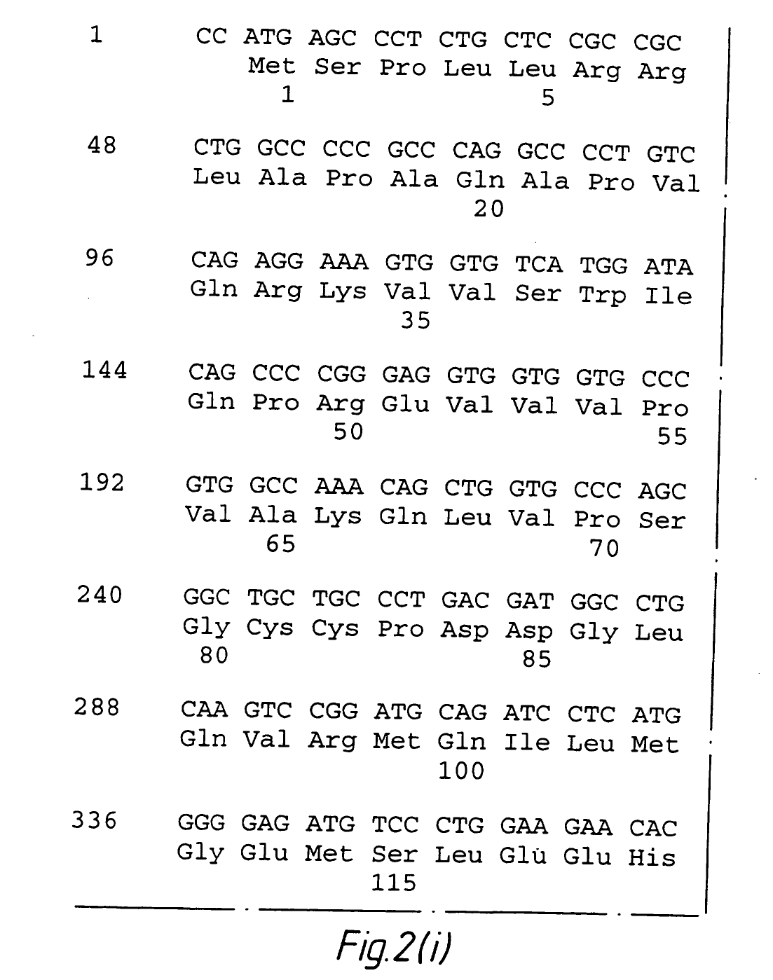

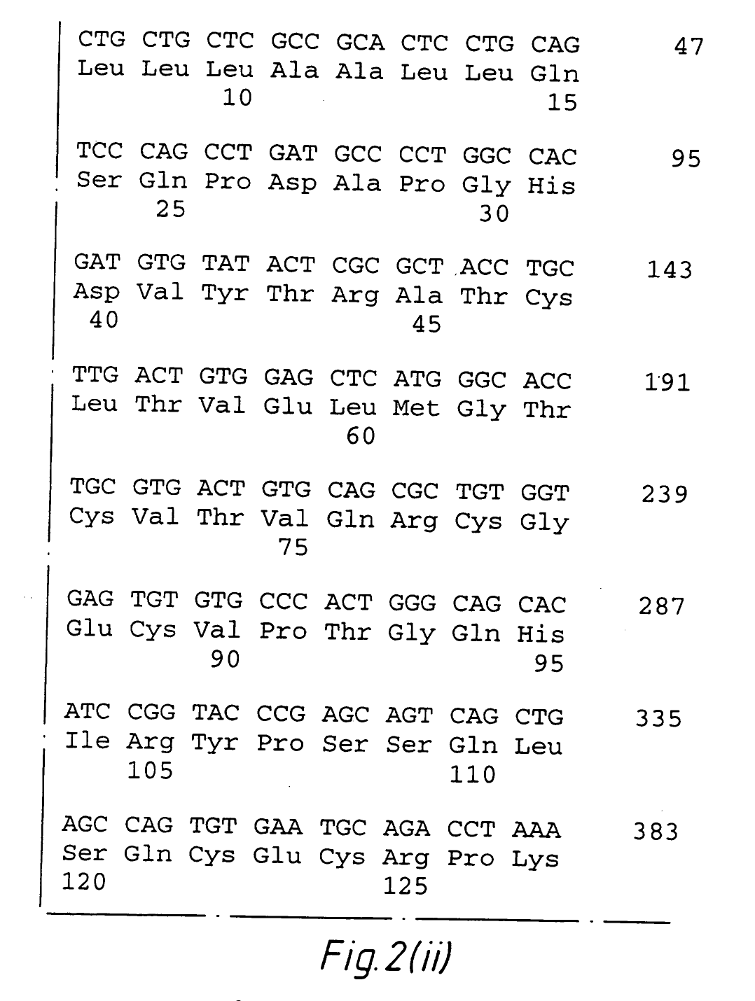

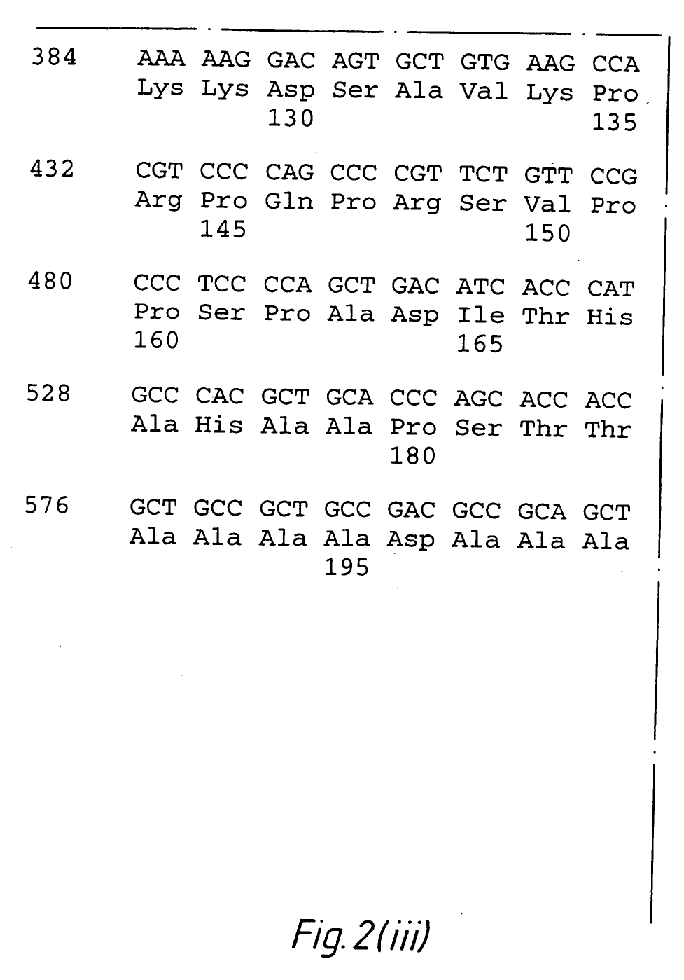

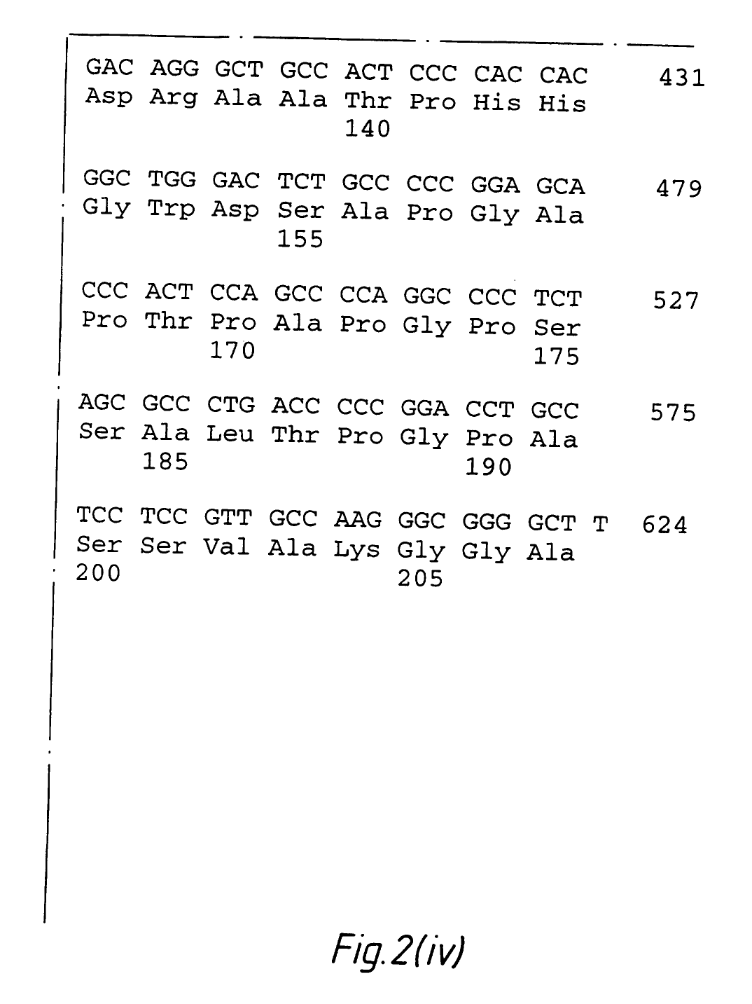

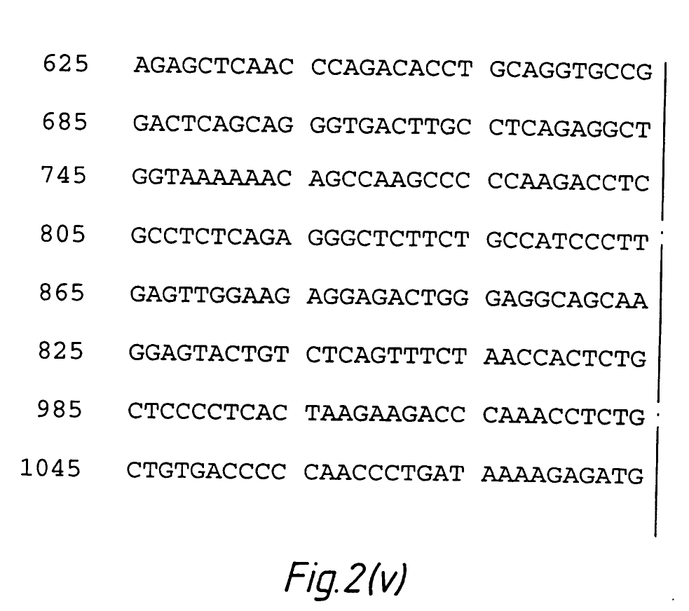

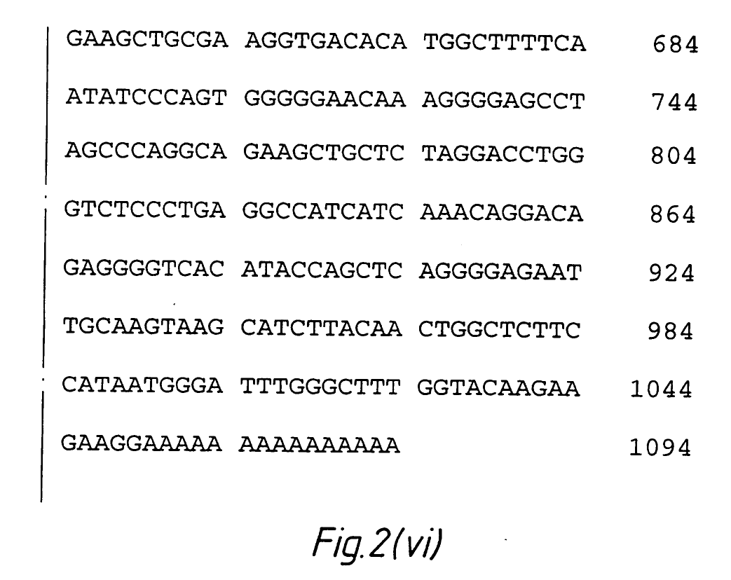

FIG. 2 Nucleotide sequence [SEQ ID NO:3] and corresponding amino acid sequence [SEQ ID NO:4] of SOM175.

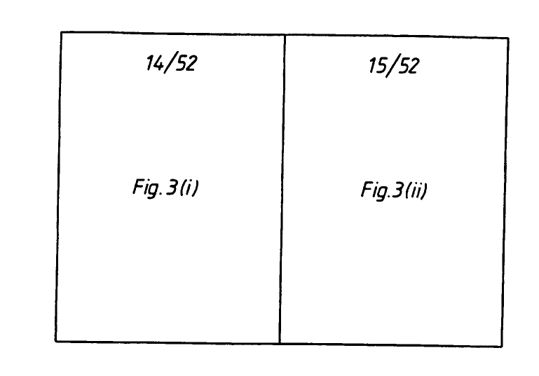

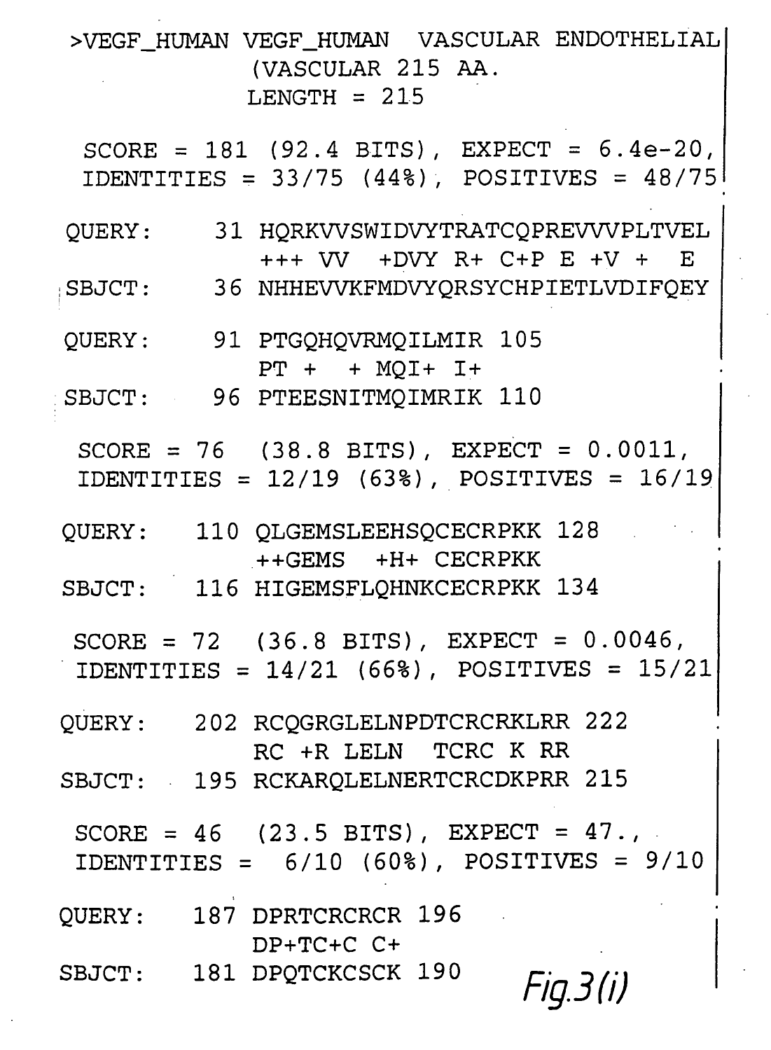

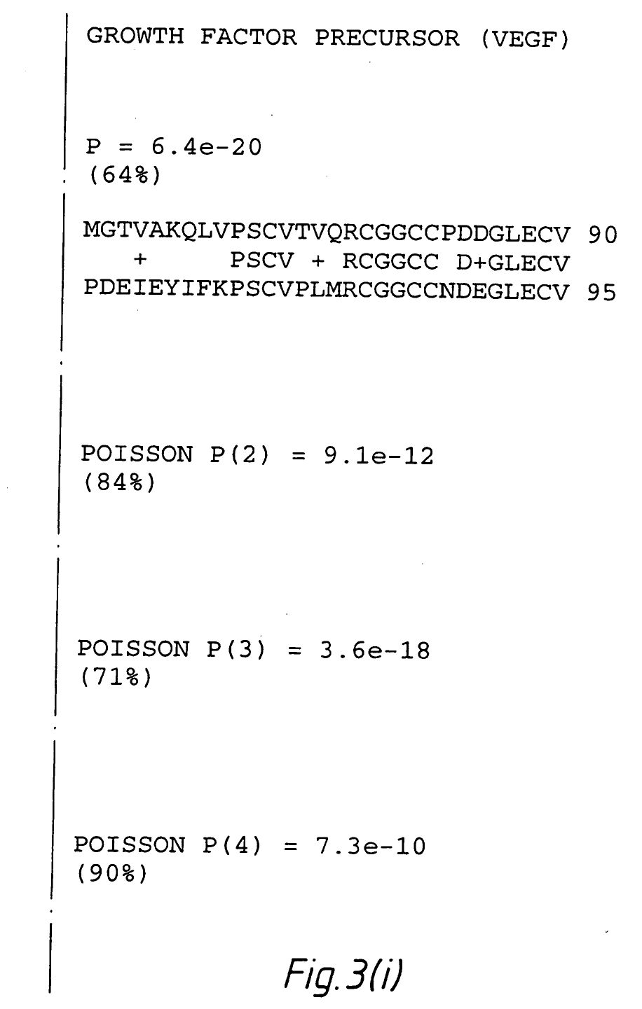

FIG. 3 Results of BLAST search with SOM175 protein sequence.

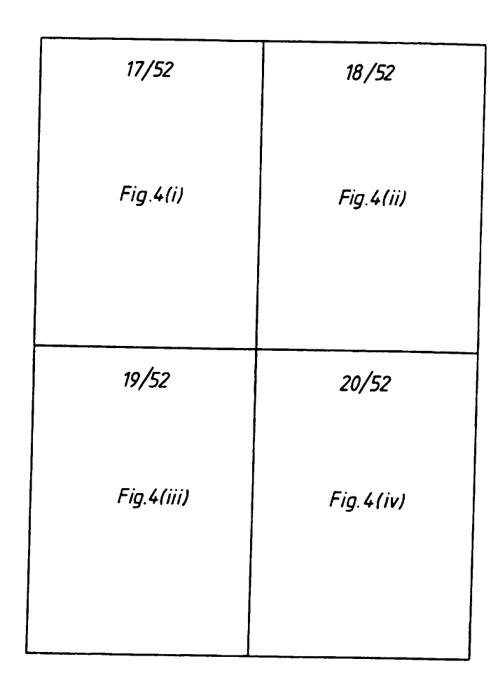

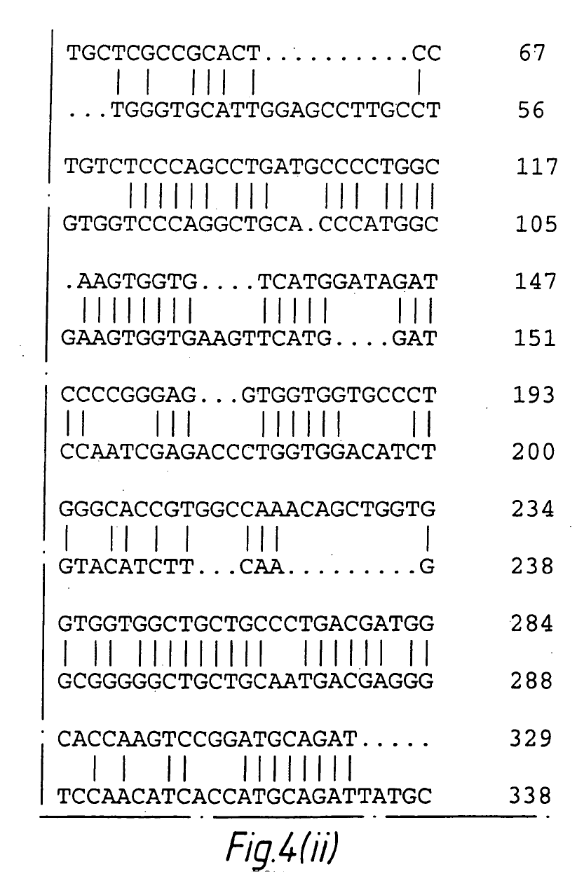

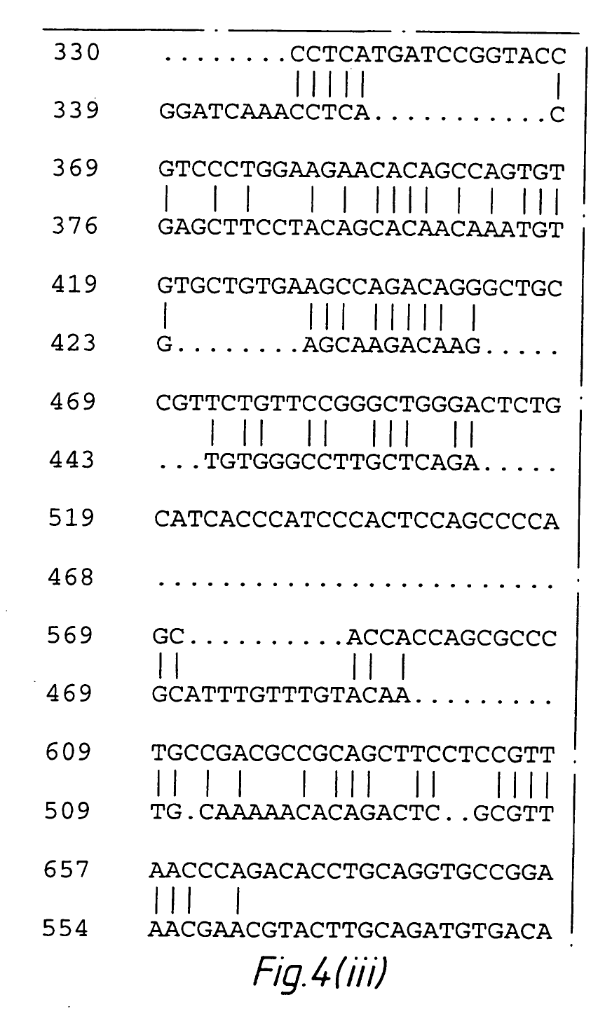

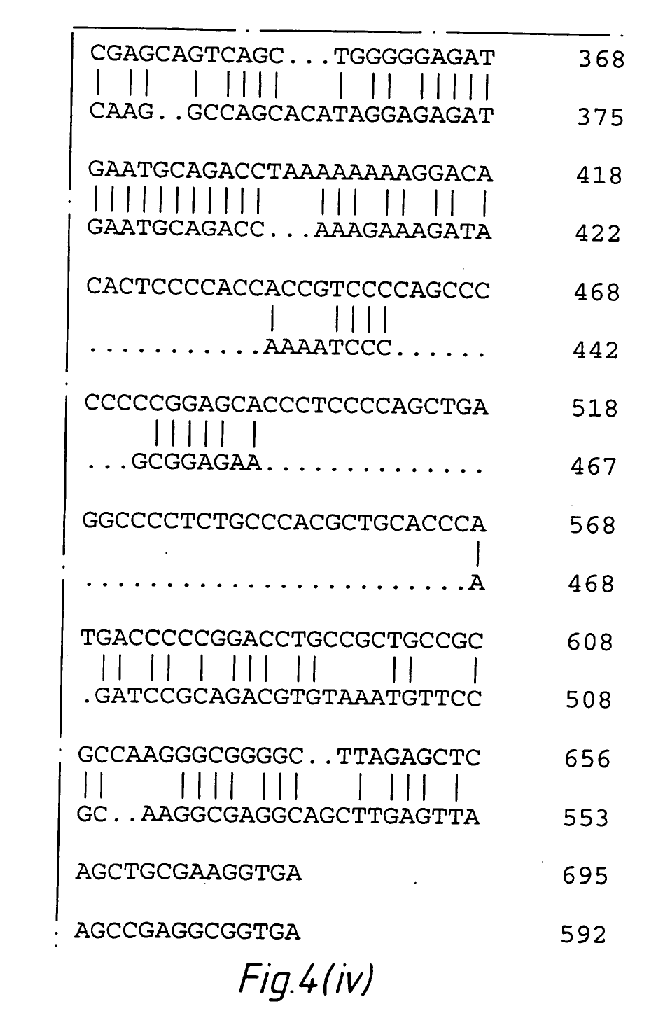

FIG. 4 BESTFIT alignment of VEGF cDNA and SOM175 cDNA.

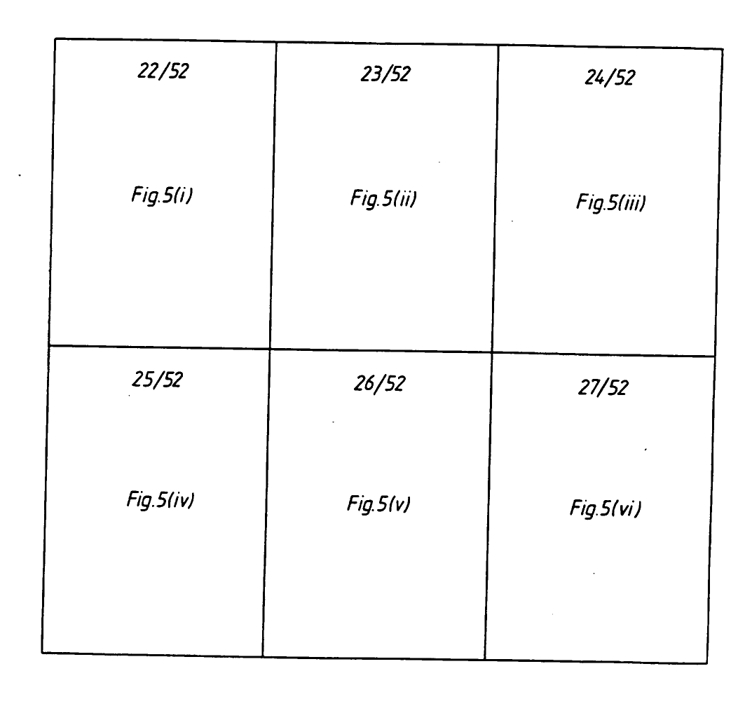

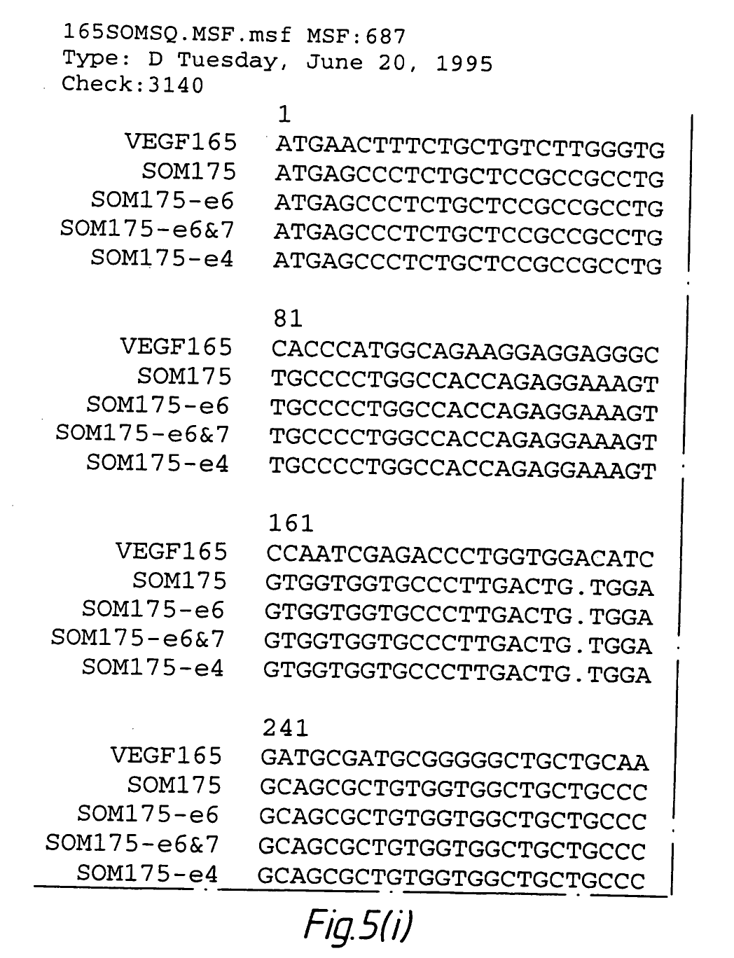

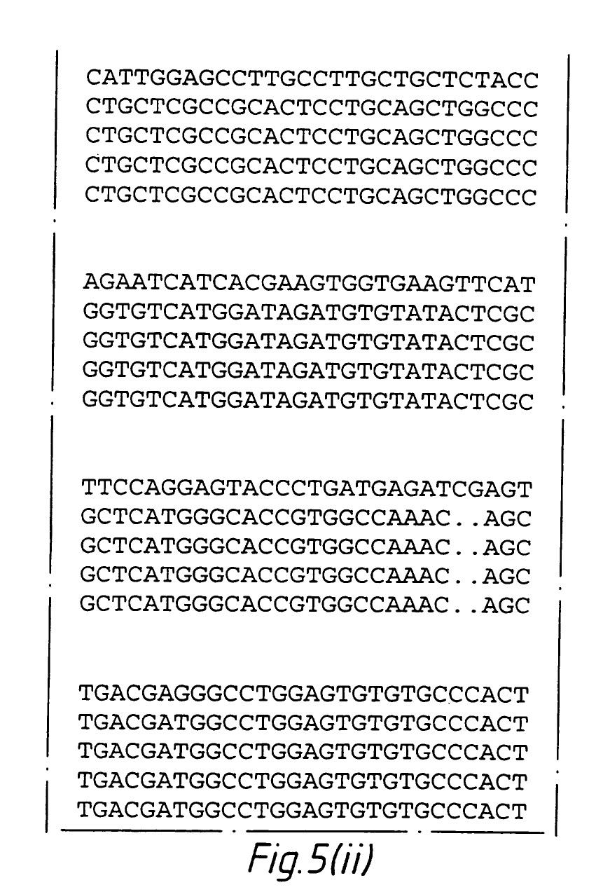

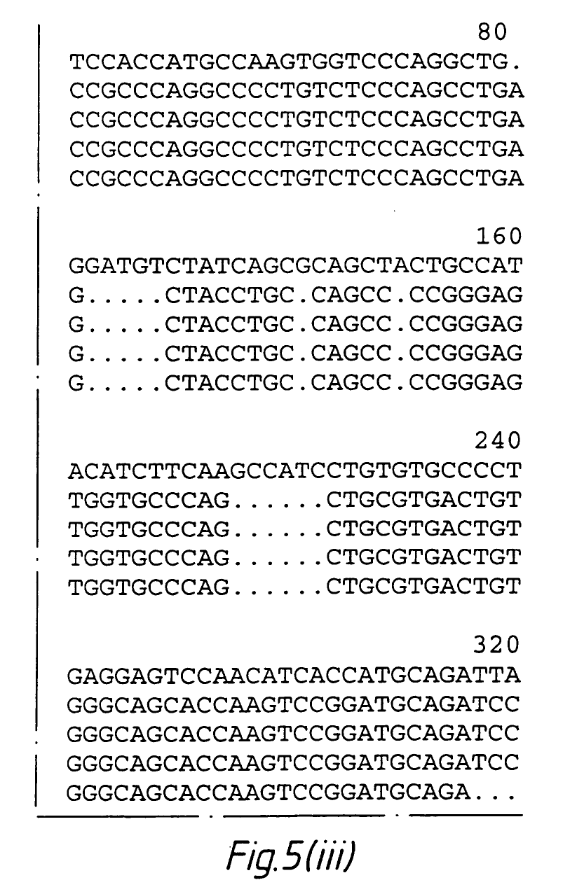

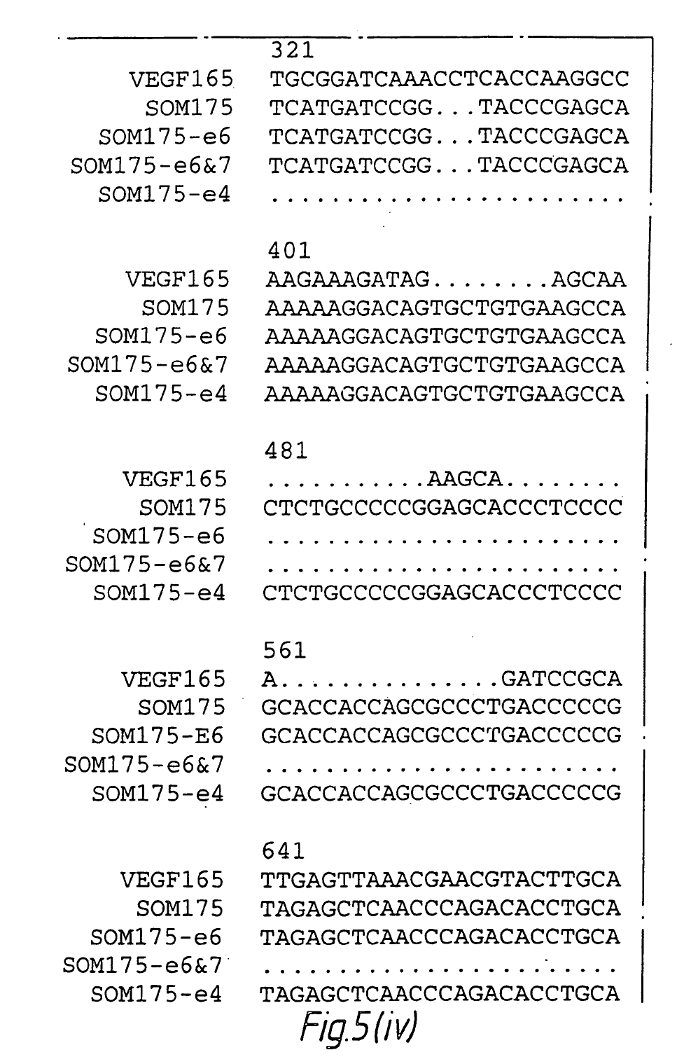

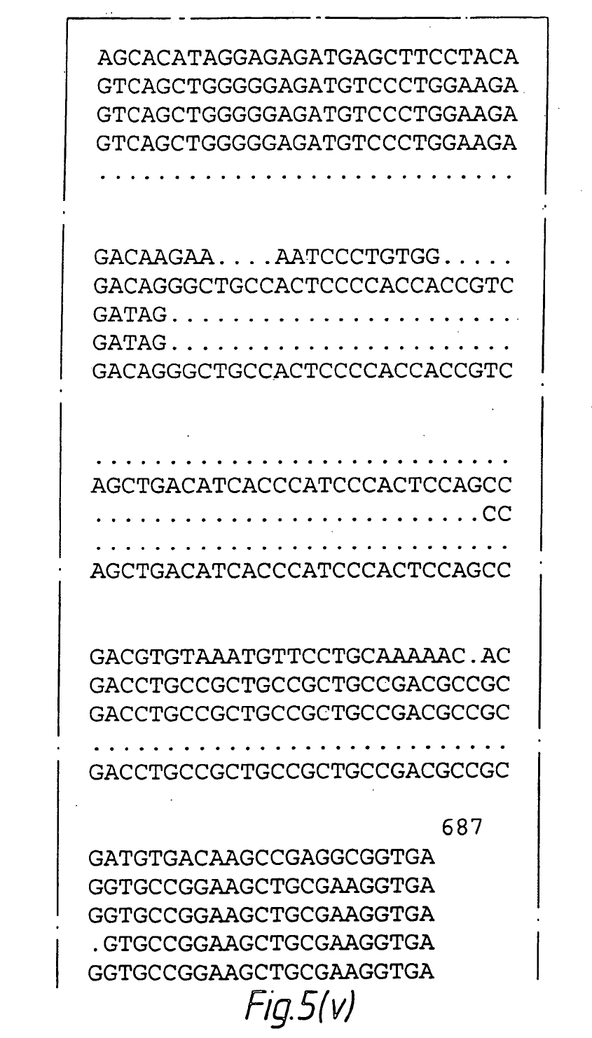

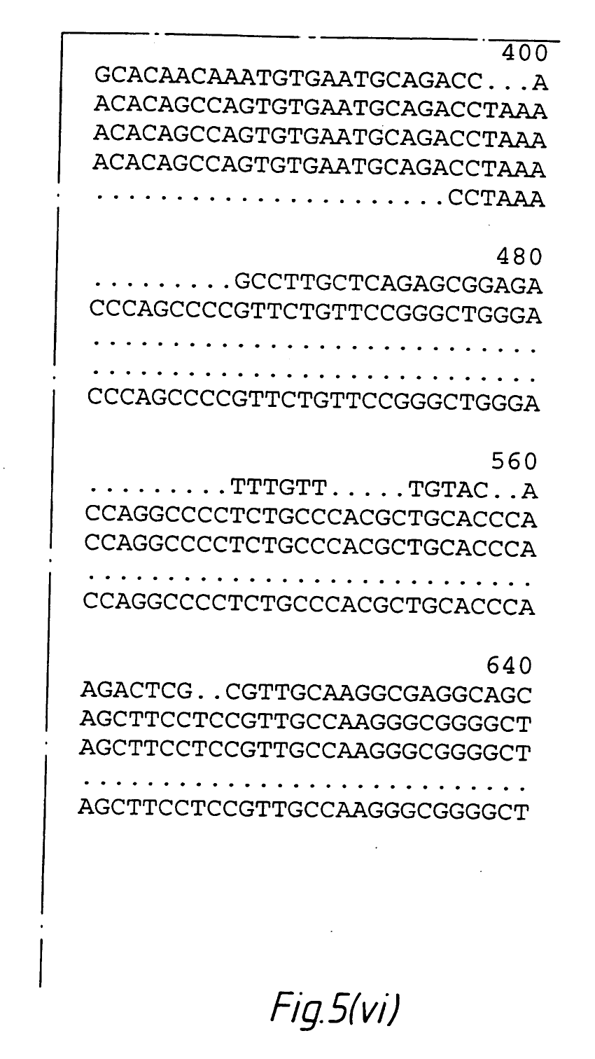

FIG. 5 Multiple alignment of VEGF₁₆₅ with SOM175 and its splice variants at the nucleotide level.

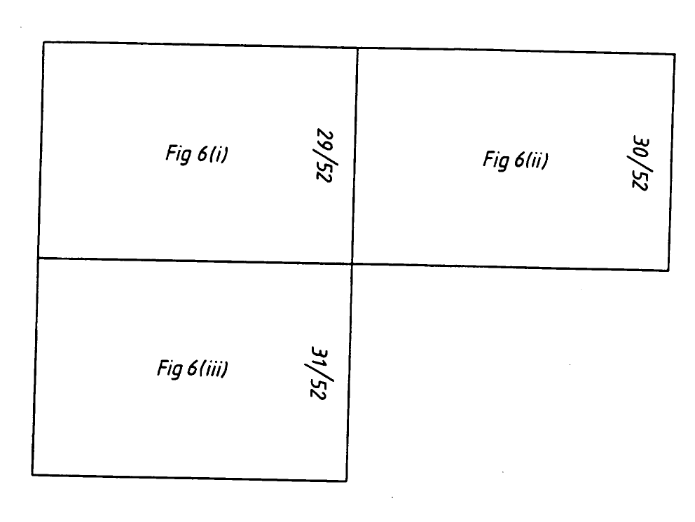

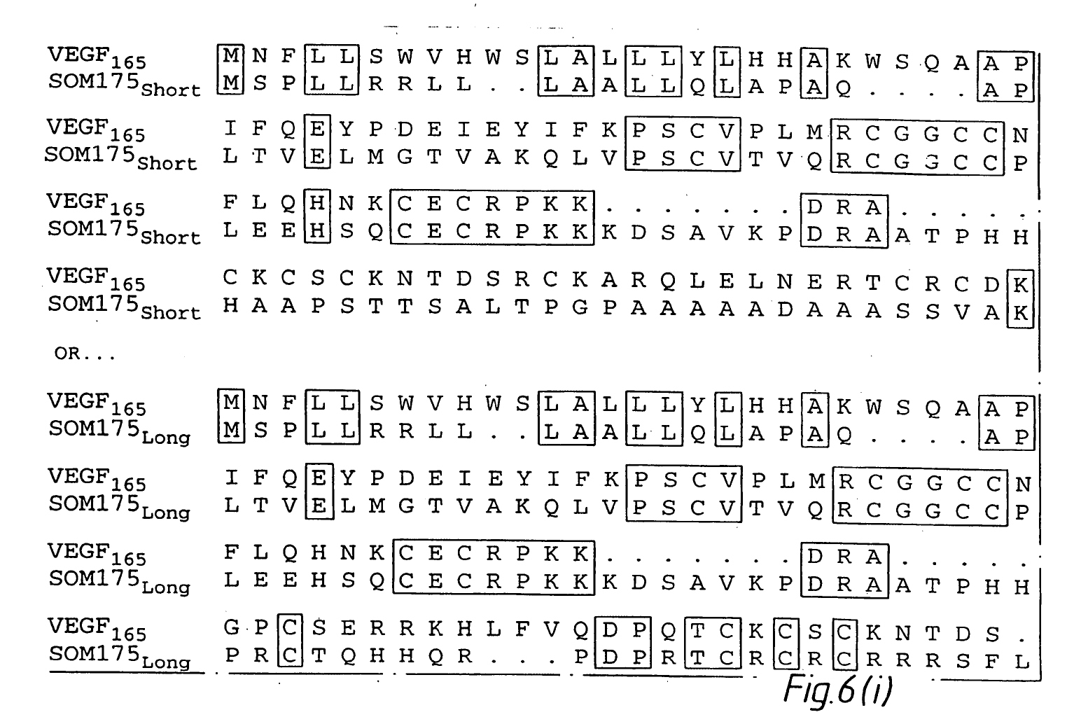

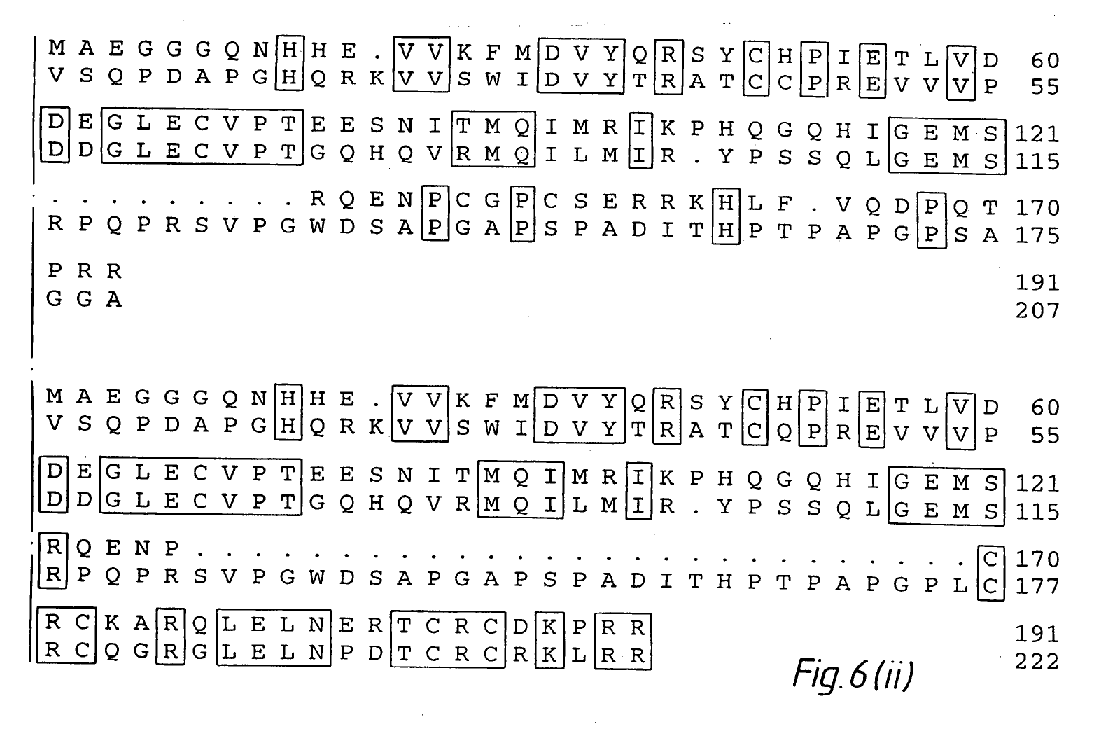

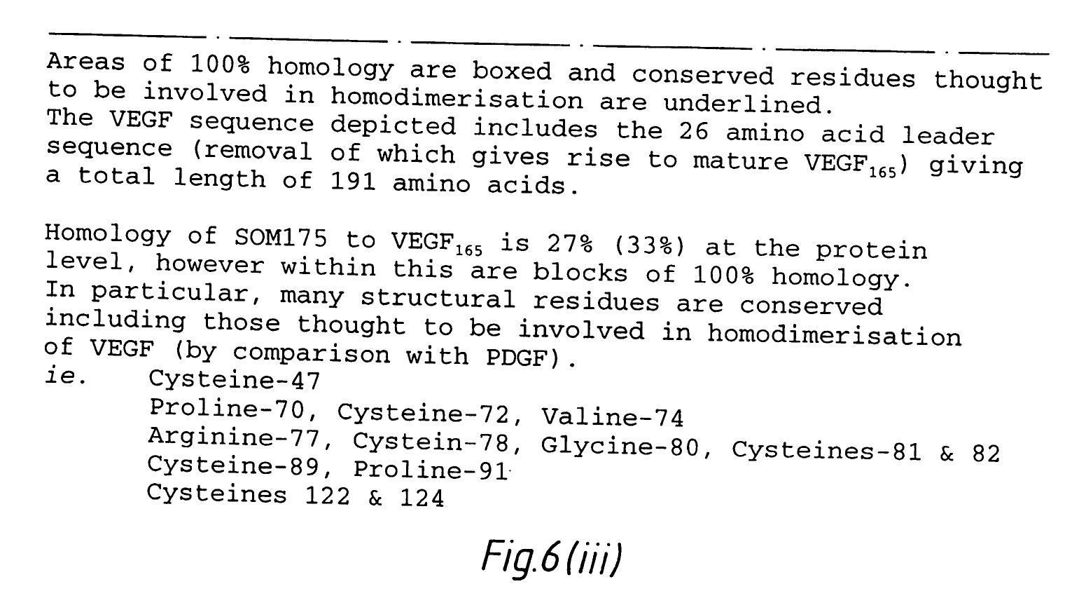

FIG. 6 Multiple alignment of VEGF₁₆₅ with SOM175 and its splice variants at the amino acid level.

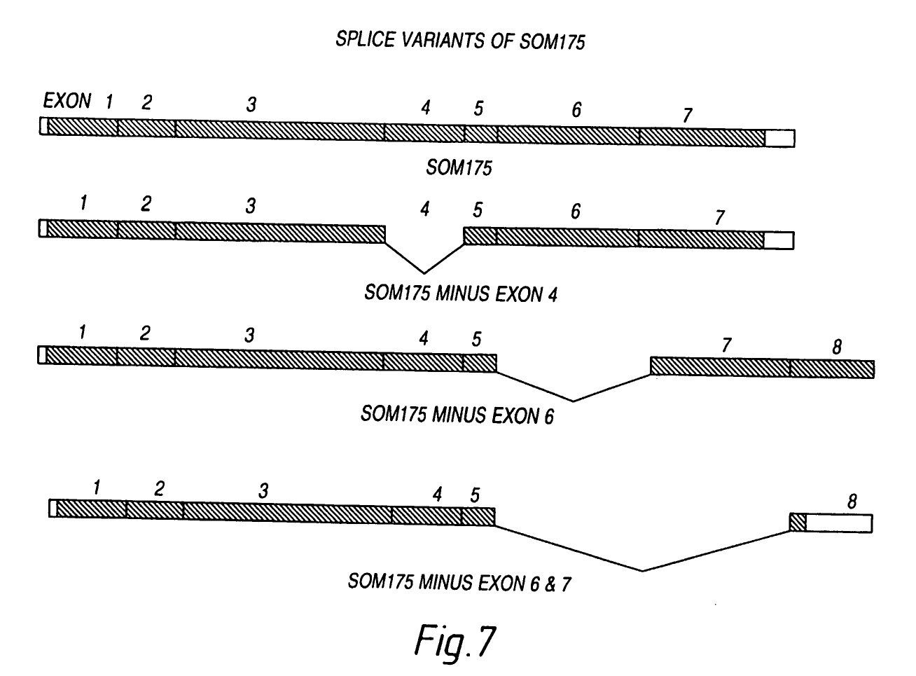

FIG. 7 Diagrammatic representation of SOM175 and its splice variants.

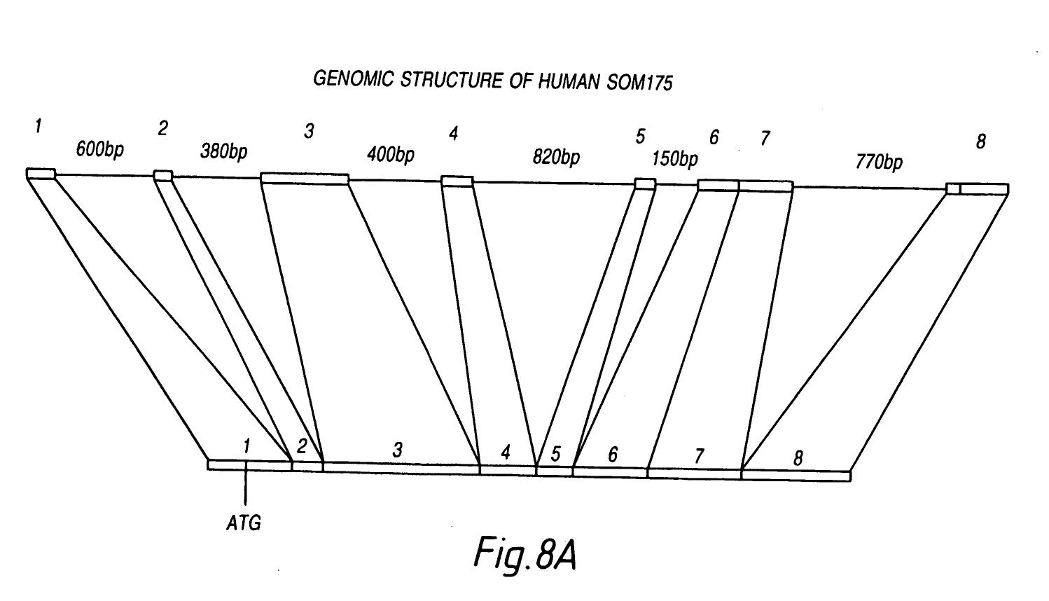

FIG. 8(a) Diagrammatic representation of genomic structure of human SOM175 genomic showing exon/intron map.

FIG. 8(b) Diagrammatic representation of genomic structure of human SOM175 showing exon/intron boundaries.

FIG. 9 Nucleotide and predicted peptide sequences derived from mVRF cDNA clones. Numbering of nucleotides are given on the left, starting from the A of the initiation codon. Amino acids are numbered on the right, starting from the first residue of the predicted mature protein after the putative signal peptide has been removed. The alternately spliced region is double underlined and the resulting peptide sequence from each mRNA is included. A potential polyadenylation signal is indicated in boldface. Start and stop codons of mVRF₁₆₇ and mVRF₁₈₆ are underlined and a polymorphic AC repeat in the 3′ UTR is indicated by a stippled box. The positions of intron/exons boundaries are indicated by arrowheads.

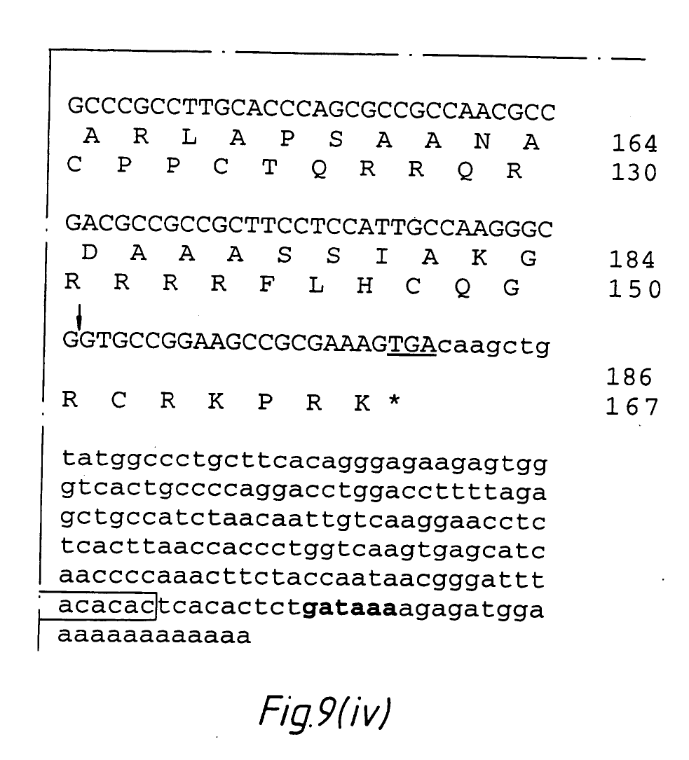

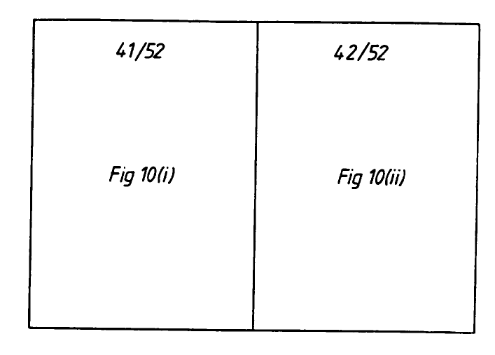

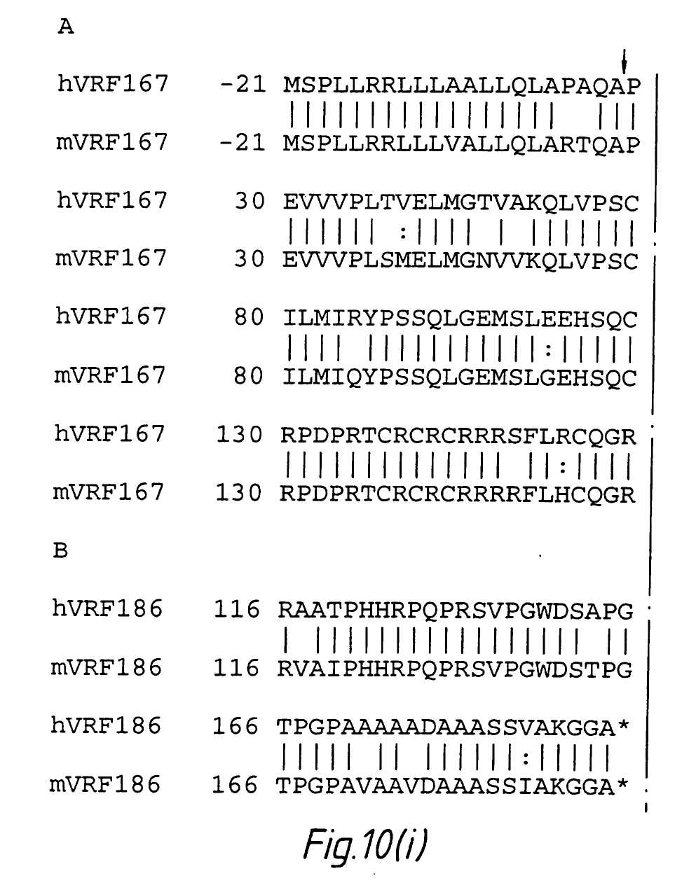

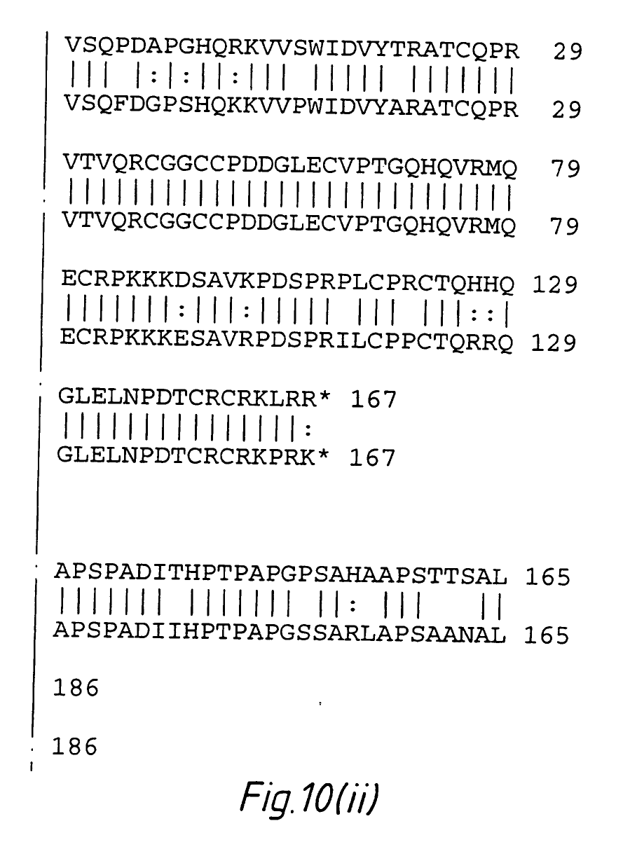

FIG. 10 BESTFIT alignments of human and murine VRF protein isoforms. A: mVRF₁₆₇ and hVRF₁₆₇. B: mVRF₁₈₆ and hVRF₁₈₆ from the point where the sequences diverge from the respective 167 amino acid isoforms. Amino acid identities are marked with vertical bars and conserved amino acids with colons. An arrow marks the predicted signal peptide cleavage site of human and mouse VRF.

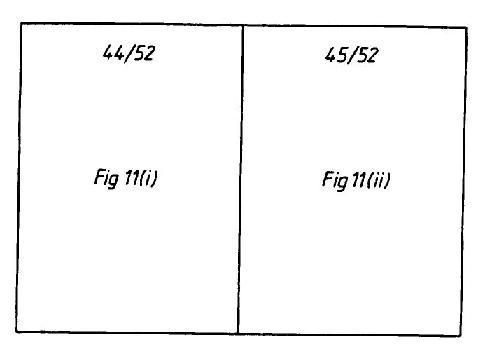

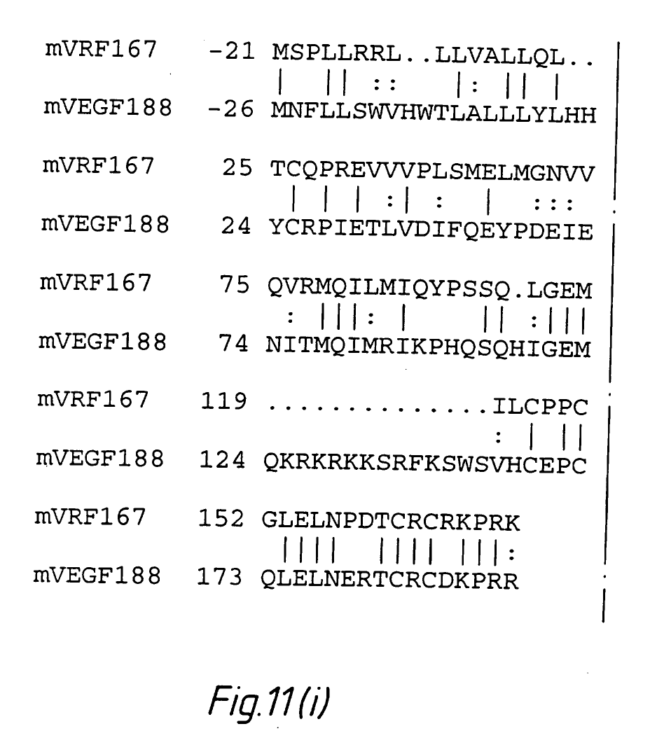

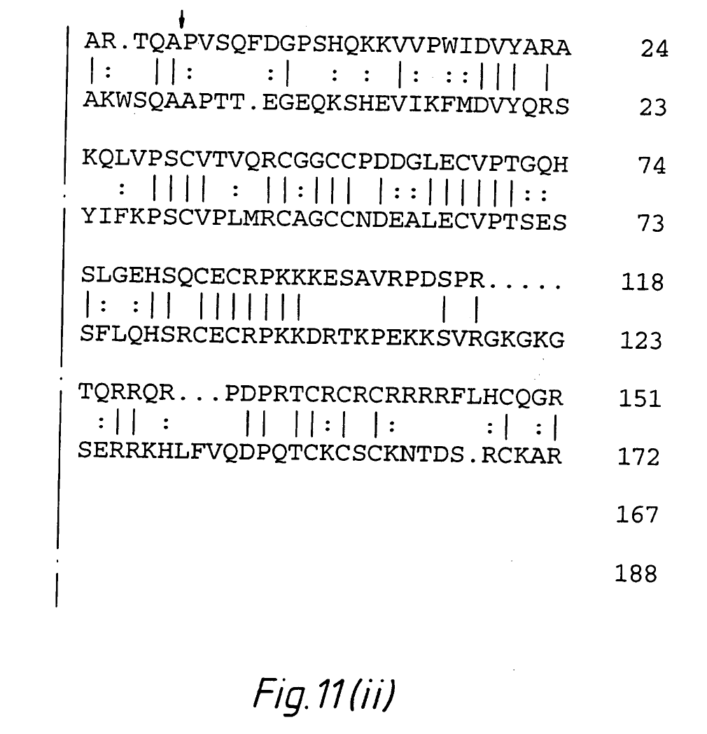

FIG. 11 BESTFIT alignment of mVRF₁₆₇ and mVEGF₁₈₈ (Breier et al, 1992) peptide sequences. An arrow marks the signal peptide cleavage site of mVEGF. Identical amino acids are indicated by vertical bars and conservative substitutions by colons. Numbering of amino acids is as described in the legend to FIG. 9.

FIG. 12 Comparison of gene structure between VRF (a generic VRF gene is shown since the intron/exon organisation of the mouse and human homologues is almost identical) and other members of the human VEGF/PIGF/PDGF gene family. Exons are represented by boxes. Protein coding regions and untranslated regions are shown by filled and open sections respectively. The hatched region in VRF indicates the additional 3′ UTR sequence formed by alternate splicing of the VRF₁₈₆ isoform. Potential alternate splice products of each gene are shown.

FIG. 13 Autoradiogram of a Northern blot of total RNA from various adult mouse tissues (as indicated) hybridised with an mVRF cDNA clone. A major transcript of 1.3 kb was detected in all samples.

FIG. 14 Film autoradiographs (A-C) and dark-field micrographs (D-E) illustrating the expression pattern of mVRF and mRNA in the mouse. In the E14 mouse embryo (A) positive signals are present over the developing heart (Ha) and cerebral cortex (Cx). A low background signal is also present over other tissues in the section. In the E17 embryo (B) and the heart (Ha) is clearly visible due to a strong hybridisation signal. An equally strong signal is present over brown adipose tissue (Fa) in the back and around the thoracic cage. A moderate hybridisation signal is present over the spinal cord (SC) and the tongue (T). The background signal is reduced compared with the E14 embryo. In the young adult mouse (C-D), positive signals are present over the heart (Ha) and adipose tissue (Fa) around the thoracic cage, while, for example, the lungs (Lu) are unlabeled). The hybridisation signal over the heart is evenly distributed over the entire left ventricle, including papillary muscles (D). In the E17 heart hybridised with an excess of cold probe, no positive signal is present (E). Scale bars=0.5 mm (A), 1.2 mm (B), 1 mm (C), 0.3 mm (D), 0.1 mm (E).

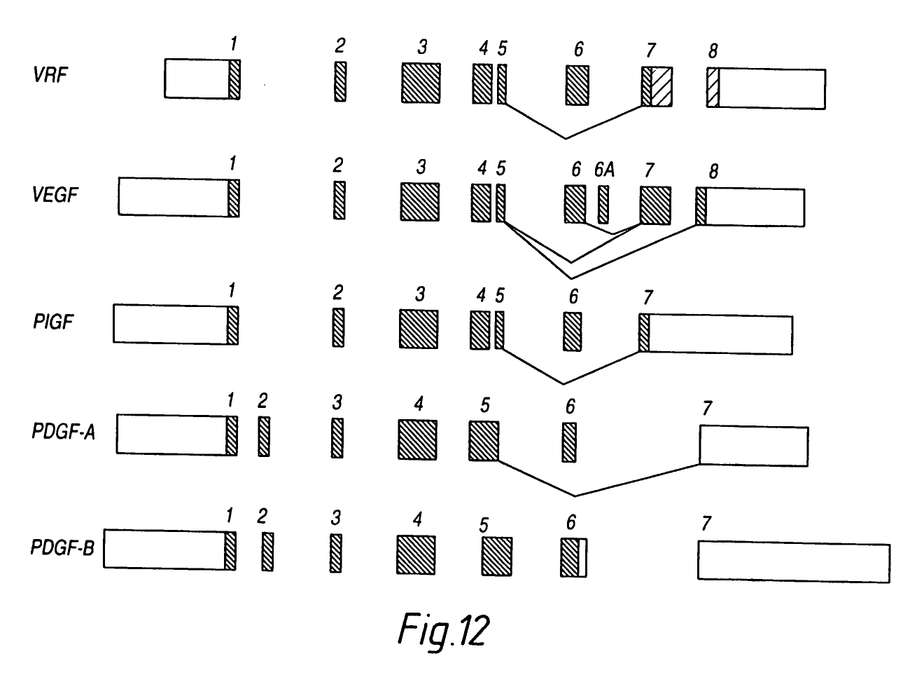

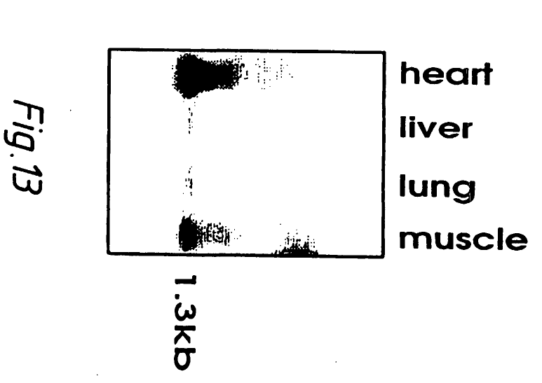

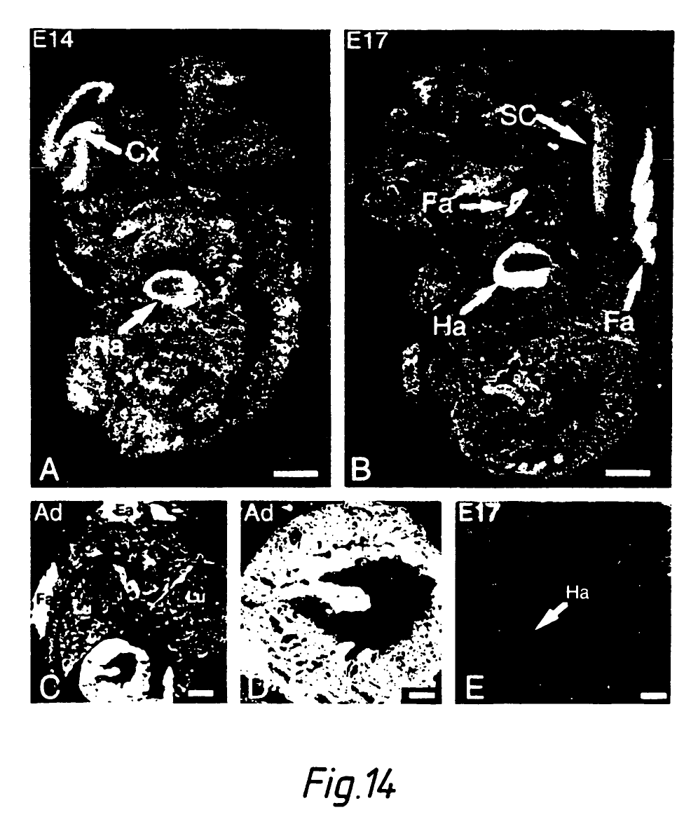

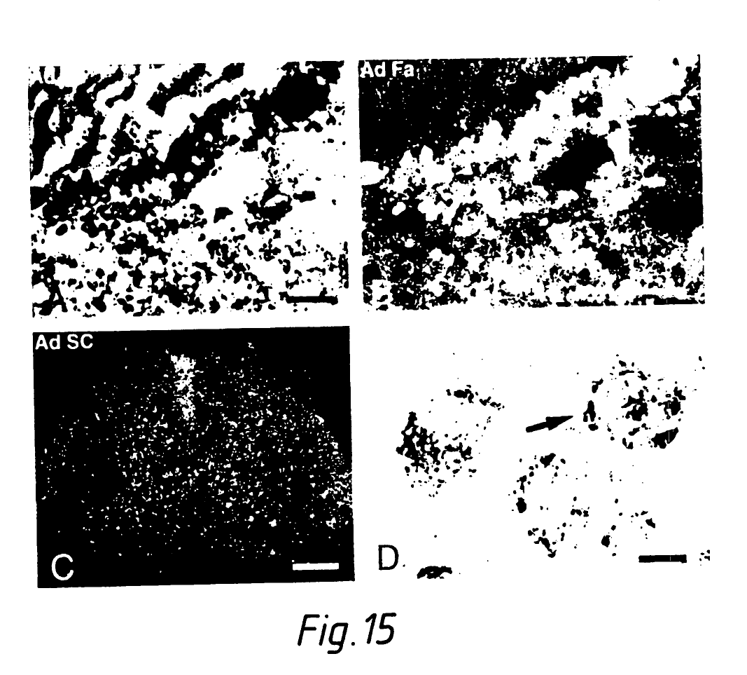

FIG. 15 Dark- (A and C) and bright-field (B and D) micrographs showing mVRF mRNA expression in mouse adipose tissue (A-B) and spinal cord (C-D). A strong hybridisation signal is present over fat (A), as shown by the strong labeling in Sudan black stained sections (B). A weak signal is present also in skeletal muscle (M in A-B). In the adult spinal cord (C) the mVRF probes gave a neuronal staining pattern over the gray matter. Toloudine counterstaining showing that motoneurons in the ventral horn (D), interneurons in the deep part of the dorsal horn and around the central canal (not shown) where largely positive for mVRF mRNA. Scale bars=0.1 mm (A), 0.1 mm (B), 0.25 mm (C), 0.015 mm (D).

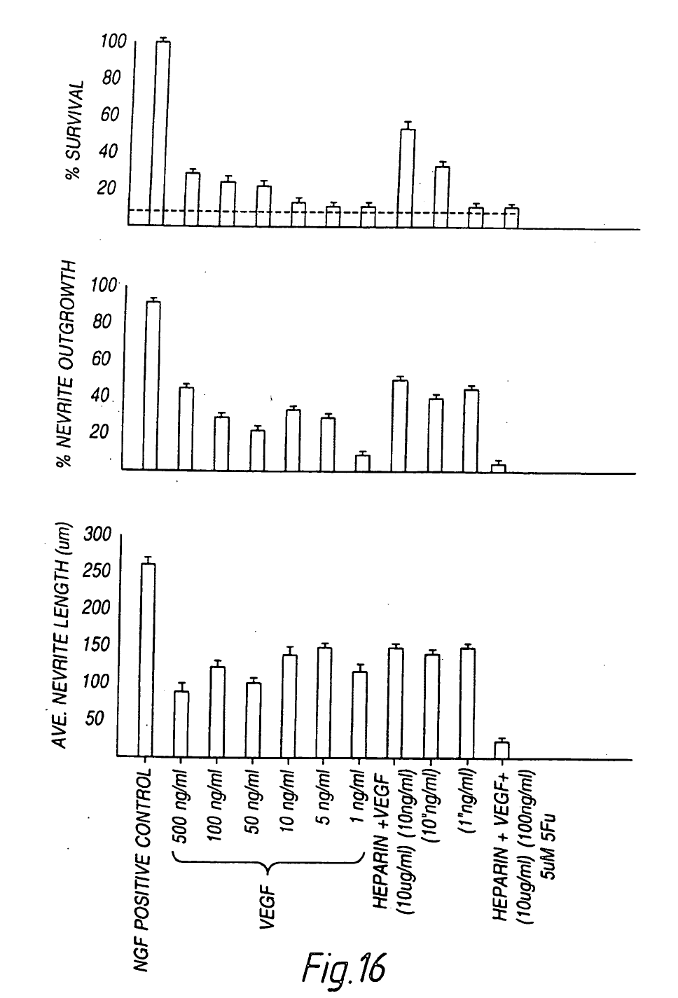

FIG. 16 Effect of VEGF on embryonic day 8 (E8) chick sensory neurons as determined by % survival, % neurite outgrowth and average neurite length (μm).

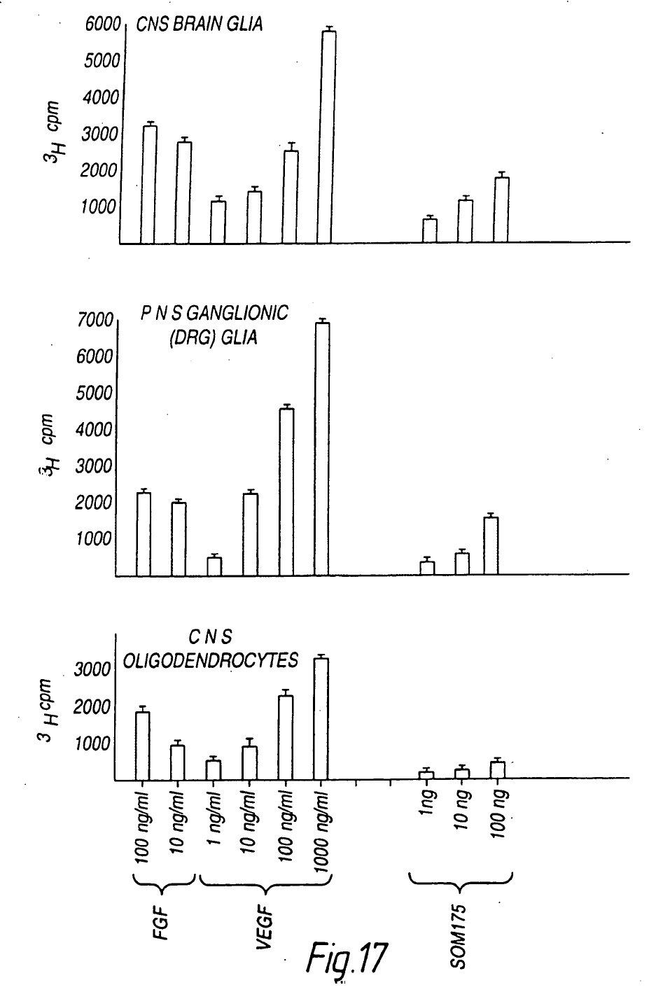

FIG. 17 Effects of VEGF and SOM175 on chick glia. Tested were CNS glial, peripheral glia and CNS oligodendrocytes.

FIG. 18 Effect of various SOM175 proteins on mouse astroglial cells. ▪ ³H (cpm)
1. FGF-2 (10 ng/ml) positive control
2. SOMΔX6* 1 ng/ml
   *This refers to SOM175 absent exon 6;
3. SOMΔX6 10 ng/ml
4. SOMΔX6 100 ng/ml
5. SOMΔX6 1000 ng/ml
6. SOMΔX6 1000 ng/ml, no heparin
7. SOMX6** 1 ng/ml
   **This refers to SOM175.
8. SOMX6 10 ng/ml
9. SOMX6 100 ng/ml
10. SOMX6 1000 ng/ml
11. SOMX6 1000 ng/ml, no heparin

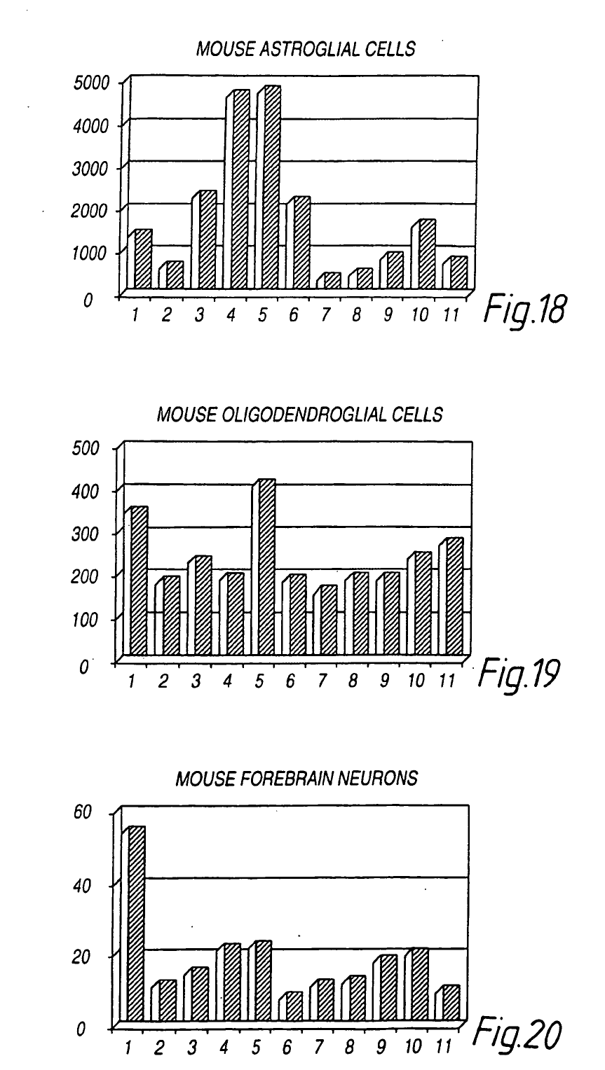

FIG. 19 Effect of various SOM175 proteins on mouse oligodendroglial cells. ▪ ³H (cpm)
1. FGF-2 (10 ng/ml) positive control
2. SOMΔX6* 1 ng/ml
   *This refers to SOM175 absent exon 6;
3. SOMΔX6 10 ng/ml
4. SOMΔX6 100 ng/ml
5. SOMΔX6 1000 ng/ml
6. SOMΔX6 1000 ng/ml, no heparin
7. SOMX6** 1 ng/ml
   ** This refers to SOM175.
8. SOMX6 10 ng/ml
9. SOMX6 100 ng/ml
10. SOMX6 1000 ng/ml
11. SOMX6 1000 ng/ml, no heparin

FIG. 20 Effect of various SOM175 proteins on mouse forebrain neurons. ▪ % survival
1. FGF-2 (10 ng/ml) positive control
2. SOMΔX6* 1 ng/ml
   *This refers to SOM175 absent exon 6;
3. SOMΔX6 10 ng/ml
4. SOMΔX6 100 ng/ml
5. SOMΔX6 1000 ng/ml
6. SOMΔX6 1000 ng/ml, no heparin
7. SOMX6** 1 ng/ml
   **This refers to SOM175.
8. SOMX6 10 ng/ml
9. SOMX6 100 ng/ml
10. SOMX6 1000 ng/ml

11. SOMX6 1000 ng/ml, no heparin

TABLE 1
SUMMARY OF SEQUENCE IDENTITY NUMBERS

| | |
|---|---|
| SEQ ID NO: 1 | Nucleotide sequence of VEGF₁₆₅ |
| SEQ ID NO: 2 | Amino acid sequence of VEGF₁₆₅ |
| SEQ ID NO: 3 | Nucleotide sequence of SOM175 (VEGF-like |
| | molecules) |
| SEQ ID NO: 4 | Amino acid sequence of SOM175 |
| SEQ ID NO: 5 | Nucleotide sequence of SOM175 absent exon 6 |
| SEQ ID NO: 6 | Amino acid sequence of SOM175 absent exon 6 |
| SEQ ID NO: 7 | Nucleotide sequence of SOM175 absent exon 6 and |
| | exon 7 |
| SEQ ID NO: 8 | Amino acid sequence of SOM175 absent exon 6 and |
| | exon 7 |
| SEQ ID NO: 9 | Nucleotide sequence of SOM175 absent exon 4 |
| SEQ ID NO: 10 | Amino acid sequence of SOM175 absent exon 4 |
| SEQ ID NO: 11 | Oligonucleotide |
| SEQ ID NO: 12 | Oligonucleotide |
| SEQ ID NO: 13 | Oligonucleotide |
| SEQ ID NO: 14 | Oligonucleotide |

### EXAMPLE 1

Human cDNA Clones

The original SOM175 cDNA was isolated by screening a human foetal brain library (λzapII, Stratagene) with the cosmid D11S750 (Larsson et al, 1992). The plasmid was excised “in vivo” and a single 1.1 kb cDNA was obtained. Three independent SOM175 cDNAs clones were also isolated from a human foetal spleen library (Strategane, Uni-zap) using the above-mentioned SOM175 insert as a probe. Three clones were obtained: SOM175-4A, -5A and -6A. SOM175-5A is an alternately spliced clone with exon 4 being absent (SOM175-e4). These library screens were performed using hybridisation conditions recommended by the manufacturer of the library (Stratagene) and random primed insert of SOM175.

Two partial human SOM175 cDNAs have also isolated from a λGT11 human melanoma cell line A2058 library (Clontech) cDNA library screens were performed using hybridisation conditions described by Church and Gilbert, 1984). In each case, the probe was generated by random priming of a PCR product derived from SOM175 (18f-700r).

Mouse cDNA Clones

Human SOM175 was also used to screen a mouse neonatal whole brain cDNA library (Unizap, Stratagene). Four non-chimeric clones were isolated: M175-A, B, C, D. All clones were partial cDNAs and M175-C contained several introns. Three of these cDNAs lacked the exon 6.

Another clone referred to as M1 was completely sequenced and was found to contain the full open reading frame plus part of the 5′utr and total 3′utr.

### EXAMPLE 2

### DNA Sequence Analysis

The entire sequence of the cDNA clone (SOM175) was compiled and is shown in FIG. 2 with its corresponding amino acid sequence. This sequence was screened for open reading frames using the MAP program (GCG, University of Wisconsin). A single open reading frame of 672 bp was observed (see FIG. 2). There appears to be little 5′ untranslated sequences (2 bp). The 3′ untranslated region appears to be complete as it includes a poly-adenylation signal and poly-A tail.

Database homology searches were performed using the BLAST algorithm (run at NCBI, USA). This analysis revealed homology to several mammalian forms of VEGF (see FIG. 3). The amount of homology between SOM175 and human VEGF₁₆₅ was determined using the BESTFIT program (GCG, University of Wisconsin; see FIGS. 4 and 5). Nucleotide homology was estimated at 69.7% and protein homology was estimated as at least 33.3% identity and 52.5% conservation using BESTFIT analysis. BLAST analysis on nucleotide sequences revealed the almost complete match to a human expressed sequence tag EST06302 (Adams et al., 1993).

These data indicate that SOM175 encodes a growth factor that has structural similarities to VEGF. Both genes show start and stop codons in similar positions and share discrete blocks of homology. All 8 cysteines as well as a number of other VEGF residues believed to be involved in dimerisation are conserved. These residues are Cysteine-47, Proline-70, Cysteine-72, Valine-74, Arginine-77, Cysteine-78, Glycine-80, Cysteine-81, Cysteine-82, Cysteine-89, Proline-91, Cysteine-122 and Cysteine-124 and are shown in FIG. 6. Given the structural conservation between VEGF and the SOM175 gene product it is also possible that they share functional similarities. It is proposed that SOM175 encodes a VEGF-like molecule that shares some properties with VEGF but has unique properties of its own. The nucleotide sequence and corresponding amino acid sequence of VEGF₁₆₅ is shown in FIG. 1.

### EXAMPLE 3

The percentage similarity and divergence between VEGF₁₆₅ family and SOM175 family (protein) were analysed using the Clustal method, MegAlign Software, DNASTAR, Wisconsin. The results are shown in Tables 2.1 and 2.2. The alternatively spliced forms of SOM175 are abbreviated to SOM715-e6 where all of exon 6 is deleted; SOM715-e6 and 7 where all of exons 6 and 7 are deleted; and SOM175-e4 where all of exon 4 is deleted. The spliced form of SOM175 are shown in FIG. 7. Genomic maps of SOM175 showing intron/exon boundaries are shown in FIGS. 8a and 8b.

TABLE 2.1

| | | | | |
|---|---|---|---|---|
| | | SOM175- | SOM175- | SOM175- |
| VEGF₁₆₅ | SOM175 | e6 | e6&7 | e4 |

A Percent nucleotide similarity between splice variants of SOM175 and
human VEGF₁₆₅

| | | | | | |
|---|---|---|---|---|---|
| VEGF₁₆₅ | *** | 34.9 | 39.7 | 41.4 | 37.0 |
| SOM175 | | *** | 98.9 | 95.1 | 99.2 |
| SOM175-e6 | | | *** | 98.8 | 84.0 |
| SOM175- | | | | *** | 80.3 |
| e6&7 | | | | | |
| SOM175-e4 | | | | | *** |

B Percent nucleotide divergence between splice variants of SOM175 and
human VEGF₁₆₅

| | | | | | |
|---|---|---|---|---|---|
| VEGF₁₆₅ | *** | 41.7 | 41.6 | 41.7 | 41.8 |
| SOM175 | | *** | 0.2 | 0.2 | 0.0 |
| SOM175-e6 | | | *** | 0.0 | 0.2 |
| SOM175- | | | | *** | 0.3 |
| e6&7 | | | | | |
| SOM175-e4 | | | | | *** |

TABLE 2.2

| | | | | |
|---|---|---|---|---|
| | | SOM175- | SOM175- | SOM175- |
| VEGF₁₆₅ | SOM175 | e6 | e6&7 | e4 |

A Percent amino acid identity between splice variants of SOM175 and
human VEGF₁₆₅

| | | | | | |
|---|---|---|---|---|---|
| VEGF₁₆₅ | *** | 31.4 | 42.3 | 33.5 | 40.6 |
| SOM175 | | *** | 74.7 | 73.7 | 99.1 |
| SOM175-e6 | | | *** | 76.8 | 99.1 |
| SOM175- | | | | *** | 99.1 |
| e6&7 | | | | | |
| SOM175-e4 | | | | | *** |

B Percent amino acid divergence between splice variants of SOM175 and
human VEGF₁₆₅

| | | | | | |
|---|---|---|---|---|---|
| VEGF 165 | *** | 65.7 | 55.4 | 54.6 | 57.4 |
| SOM175 | | *** | 19.9 | 4.2 | 0.0 |
| SOM175-e6 | | | *** | 0.0 | 0.0 |
| SOM175- | | | | *** | 0.0 |
| e6&7 | | | | | |
| SOM175-e4 | | | | | *** |

### EXAMPLE 4

### Bioassays to Determine the Function of SOM175

Assays are conducted to evaluate whether SOM175 has similar activities to VEGF on endothelial cell function, angiogenesis and wound healing. Other assays are performed based on the results of receptor binding distribution studies.

Assays of Endothelial Cell Function

Endothelial cell proliferation. Endothelial cell growth assays as described in Ferrara & Henzel (1989) and in Gospodarowicz et al (1989).

Vascular permeability assay. This assay, which utilises the Miles test in guinea pigs, will be performed as described in Miles & Miles (1952).

Cell adhesion assay. The influence of SOM175 on adhesion of polymorphs to endothelial cells is analysed.

Chemotaxis. This is performed using the standard Boyden chamber chemotaxis assay.

Plasminogen activator assay. Endothelial cells are tested for plasminogen activator and plasminogen activator inhibitor production upon addition of SOM175 (Pepper et al (1991)).

Endothelial cell migration assay. The ability of SOM175 to stimulate endothelial cells to migrate and form tubes is assayed as described in Montesano et al (1986).

Angiogenesis Assay

SOM175 induction of an angiogenic response in chick chorioallantoic membrane is evaluated as described in Leung et al (1989).

Possible neurotrophic actions of SOM175 are assessed using the following assays:

Neurite Outgrowth Assay and Gene Induction (PC12 Cells)

PC12 cells (a phaeochromocytoma cell line) respond to NGF and other neurotrophic factors by developing the characteristics of sympathetic neurons, including the induction of early and late genes and the extension of neurites. These cells are exposed to SOM175 and their response monitored (Drinkwater et al (1991); and Drinkwater et al (1993)).

Cultured Neurons from the Peripheral Nervous System (PNS)

Primary cultures of the following PNS neurons are exposed to SOM175 and monitored for any response:
sensory neurons from neural crest and dorsal root ganglia
sympathetic neurons from sympathetic chain ganglia
placode derived sensory neurons from nodose ganglia
motoneurons from spinal cord

The assays are described in Suter et al (1992) and in Marinou et al (1992).

Where an in vitro response is observed, in vivo assays for properties such as uptake and retrograde transport are performed as described in Hendry et al (1992).

Nerve Regeneration (PNS)

Where neurotrophic effects of SOM175 are observed, its possible role in the regeneration of axotomised sensory neurons, sympathetic neurons and motoneurons is analysed by the methods of Otto et al (1989); Yip et al (1984) and Hendry et al (1976).

Actions of SOM175 on CNS Neurons

The ability of SOM175 to promote survival of central nervous system neurons is analysed as described in Hagg et al (1992); Williams et al (1986); Hefti (1986) and Kromer (1987).

Wound Healing

The ability of SOM175 to support wound healing are tested in the most clinically relevant model available, as described in Schilling et al (1959) and utilised by Hunt et al (1967).

The Haemopoietic System

A variety of in vitro and in vivo assays on specific cell populations of the haemopoietic system are available and are outlined below:

Stem Cells

Murine

A variety of novel in vitro murine stem cell assays have been developed using FACS-purified cells:

(a) Repopulating Stem Cells

These are cells capable of repopulating the bone marrow of lethally irradiated mice, and have the Lin-, Rh^{hi}, Ly-6A/E⁺, c-kit⁺ phenotype. The test substance is tested on these cells either alone, or by co-incubation with multiple factors, followed by measurement of cellular proliferation by ³H thymidine incorporation.

(b) Late Stage Stem Cells

These are cells that have comparatively little bone marrow repopulating ability but can generate D13 CFU-S. These cells have the Lin⁻, Rh^{hi}, Ly-6A/E⁺, c-kit⁺ phenotype. The test substance is incubated with these cells for a period of time, injected into lethally irradiated recipients, and the number of D13 spleen colonies enumerated.

(c) Progenitor-Enriched Cells

These are cells that respond in vitro to single growth factors, and have the Lin⁻, Rh^{hi}, Ly-6A/E⁺, c-kit⁺ phenotype. This assay will show if SOM175 can act directly on haemopoietic progenitor cells. The test substance is incubated with these cells in agar cultures, and the number of colonies enumerated after 7-14 days.

Atherosclerosis

Smooth muscle cells play a crucial role in the development or initiation of atherosclerosis, requiring a change in their phenotype from a contractile to a synthetic state. Macrophages, endothelial cells, T lymphocytes and platelets all play a role in the development of atherosclerotic plaques by influencing the growth and phenotypic modulations of smooth muscle cell. An in vitro assay that measures the proliferative rate and phenotypic modulations of smooth muscle cells in a multicellular environment is used to assess the effect of SOM175 on smooth muscle cells. The system uses a modified Rose chamber in which different cell types are seeded onto opposite coverslips.

Effects of SOM175 on Bone

The ability of SOM175 to regulate proliferation of osteoblasts is assayed as described in Lowe et al (1991). Any effects on bone resorption are assayed as described in Lowe et al (1991). Effects on osteoblast migration and changes in intracellular molecules (e.g. cAMP accumulation, alkaline phosphatase levels) are analysed as described in Midy et al (1994).

Effects on Skeletal Muscle Cells

Effects of SOM175 on proliferation of myoblasts and development of myotubes can be determined as described by Ewton et al (1980) and by Gospodarowicz et al (1976).

### EXAMPLE 5

### Cloning Murine VEGF DNA

Isolation of cDNAs

Murine VRF (mVRF) clones were selected from a lambda Zap new born whole brain cDNA library (Stratagene). Primary phage from high density filters (5×10⁴ pfu/plate) were identified by hybridisation with a 682 bp ³²P-labelled probe generated by PCR from an hVRF cDNA (pSOM175) as described above. Hybridisation and stringent washes of nylon membranes (Hybond-N) were carried out at 65° C. under conditions described by Church and Gilbert (1984). Positive plaques were picked, purified and excised in vivo to produce bacterial colonies containing cDNA clones in pBluescript SK−.

Isolation of Genomic Clones

Genomic clones were isolated from a mouse strain SV/129 library cloned in the lambda Fix II vector (Stratagene). High density filters (5×10⁴ pfu/filter) were screened with a 563 bp ³²P-labelled probe generated by PCR amplification of the nucleotide 233-798 region of the mVRF cDNA (see FIG. 9). Positive clones were plugged and re-screened with filters containing 400-800 pfu. Large scale phage preparations were prepared using the QIAGEN lambda kit or by ZnCl₂ purification (Santos, 1991).

Nucleotide Sequencing and Analysis

cDNAs were sequenced on both strands using a variety of vector-based and internal primers with Applied Biosystems Incorporated (ABI) dye terminator sequencing kits according to the manufacturer's specifications. Sequences were analysed on an ABI Model 373A automated DNA sequencer. Peptide homology alignments were performed using the program BESTFIT (GCG, Wisconsin).

Identification of Intron/Exon Boundaries

Identification of exon boundaries and flanking regions was carried out using PCR with mouse genomic DNA or mVRF genomic lambda clones as templates. The primers used in PCR to identify introns were derived from the hVRF sequence and to allow for potential human-mouse sequence mismatches annealing temperatures 5-10° C. below the estimated Tₘ were used. All PCR products were sized by agarose gel electrophoresis and gel purified using QIAquick spin columns (Qiagen) and the intron/exon boundaries were sequenced directly from these products. In addition, some splice junctions were sequenced from subcloned genomic fragments of MVRF. Intron/exon boundaries were identified by comparing cDNA and genomic DNA sequences.

Northern Analysis

Total cellular RNA was prepared from a panel of fresh normal adult mouse tissues (brain, kidney, liver, muscle) using the method of Chomczynski and Sacchi (1987). 20 μg of total RNA were electrophoresed, transferred to a nylon membrane (Hybond N, Amersham) and hybridised under standard conditions (Church & Gilbert, 1984). Filters were washed at 65° C. in 0.1×SSC (20×SSC is 3M NaCl/0.3M trisodium citrate), 0.1% SDS and exposed to X-ray film with intensifying screens at −70° C. for 1-3 days.

Characterisation of mVRF cDNAs

Murine VRF homologues were isolated by screening a murine cDNA library with an hVRF cDNA clone. Five clones of sizes varying from 0.8-1.5 kb were recovered and sequenced. The cDNA sequences were complied to give a full length 1041 bp cDNA sequence covering the entire open reading frame (621 bp or 564 bp depending on the splice form, see below) and 3′ UTR (379 bp), as well as 163 bp of the 5′ UTR (FIG. 9).

The predicted initiation codon matched the position of the start codon in hVRF. One other out of frame ATG was located at position −47 and two termination codons were observed upstream (positions −9 and −33, respectively) and in-frame with the putative initiation codon.

The predicted N-terminal signal peptide of hVRF appears to be present in mVRF with 81% identity (17/21 amino acids). Peptide cleavage within mVRF is expected to occur after reside 21 (FIG. 10). These data suggest that mature mVRF is secreted and could therefore conceivably function as a growth factor.

As with hVRF, two open reading frames (ORFs) were detected in cDNAs isolated by library screening. Four of five clones were found to be alternatively spliced and lacked a 101 bp fragment homologous to exon 6 of hVRF. The predicted peptide sequences of the two isoforms of mVRF were determined and aligned with the corresponding human isoforms (FIG. 10).

The message encoding mVRF₁₈₆ contains a 621 bp ORF with coding sequences terminating at position +622, towards the end of exon 7 (FIG. 9). The smaller message encoding mVRF₁₆₇ actually terminates downstream of the +622 TAG site due to a frame shift resulting from splicing out of the 101 bp exon 6 and the introduction of a stop codon (TGA) at position +666, near the beginning of exon 8 (FIG. 9).

The mVRF₁₈₆ protein has strong homology to the amino and central portions of VEGF while the carboxyl end is completely divergent an is alanine rich. mVRF₁₆₇ possesses these similarities and also maintains homology to mVEGF right through to the C-terminus (FIG. 11). The overall homology of mVRF₁₆₇ to hVRF₁₆₇ was 85% identity and 92% similarity, respectively (FIG. 10). Likewise, homology between mVRF₁₆₇ and mVEGF (Breier et al, 1992) was 49% identity and 71% conservative amino acid substitution, respectively (FIG. 11).

A canonical vertebrate polyadenylation signal (AATAAA) (Birnstiel et al, 1986) was not present in the mVRF cDNA, however, the closely matching sequence GATAAA is present at similar positions in both mouse and human VRF cDNAs (FIG. 9). In contrast to hVRF, mVRF was found to contain an AC dinucleotide repeat at the extreme 3′ end of the 3′ UTR (nucleotide positions 998 to 1011, FIG. 9). Polymorphism of this repeat region was observed between some of the mVRF cDNAs, with the number of dinucleotides varying from 7 to 11.

Genomic Characterisation of mVRF

Intron/exon boundaries (Table 3) were mapped using primers which flanked sequences homologous to the corresponding hVRF boundaries. Introns I, III, IV and VI of mVRF (Table 3, FIG. 12) were smaller than the hVRF intervening sequences. The complete genomic sequence was compiled from the 5′ UTR of mVRF through to intron VI, the largest intervening region (2.2 kb), by sequencing amplified introns and cloned genomic portions of mVRF. There was only one major difference in genomic structure between mVRF and hVRF and that was the exon 7/intron VI boundary of mVRF was located 10 bp further downstream in relation to the cDNA sequence, hence exon 7 in mVRF is 10 bp longer than the corresponding exon in hVRF.

Exons 6 and 7 are contiguous in mVRF, as has been found to occur in the human homologue. The strong sequence homology between exon 6 of mVRF and hVRF (FIG. 10) suggests that this sequence is not a retained intronic sequence but rather encodes a functional part of the VRF₁₈₆ isoform.

General intron/exon structure is conserved between the various members of the VEGF gene family (VEGF, PIGF, hVRF) and therefore it is not surprising that the overall genomic organisation of the mVRF gene is very similar to these genes (FIG. 12).

Previous comparative mapping studies have shown that the region surrounding the human multiple endocrine neoplasia type 1 disease locus on chromosome 11q13 is syntenic with the proximal segment of mouse chromosome 19 (Rochelle et al, 1992).

Since the inventors have mapped the hVRF gene to within 1 kb of the human MEN1 locus (see above) it is most likely that the murine VRF gene maps near the centromere of chromosome 19.

Expression Studies of mVRF

Northern analysis of RNA from adult mouse tissues (muscle, heart, lung and liver) showed that expression appears to be ubiquitous and occurs primarily as a major band of approximately 1.3 kb in size (FIG. 14). This is somewhat different to the pattern observed for hVRF in which two major bands of 2.0 and 5.5 kb have been identified in all tissues examined. The 1.3 kb murine message presumably corresponds to the shorter of the human transcripts and the size variation thereof is most likely due to a difference in the length of the respective 5′ UTRs.

### EXAMPLE 6

### Expression of Murine VEGF in Pre- and Post-Natal Mouse

Animals

Timed pregnant (n=4) and young adult (n=2) mice (C57 inbred strain, ALAB, Sweden) were sacrificed with carbon dioxide, and the relevant tissues were taken out and frozen on a chuck. Tissues were kept at −70° C. until further use. Two gestational ages was used in this study; embryonic day 8 (E8), 14 and E17.

In situ Hybridisation Histochemistry

In situ hybridisation was performed as previously described (Dagerlind et al, 1992). Briefly, transverse sections (14 μm) were cut in a cryostat (Microm, Germany), thawed onto Probe-On slides (Fisher Scientific, USA) and stored in black sealed boxes at −70° C. until used. The sequences of the synthetic 42-mer oligonucleotides complementary to mRNA encoding mVRF were ACCACCACCTCCCTGGGCTGGCATGTGGCACGTGCATAAACG [SEQ ID NO:11] (complementary to nt 120-161) and AGTTGTTTGACCACATTGCCCATGAGTTCCATGCTCAGAGGC [SEQ ID NO:12] (complementary to nt 162-203). To detect the two alternative splice forms oligonucleotide GATCCTGGGGCTGGAGTGGGATGGATGATGTCAGCTGG [SEQ ID NO:13] (complementary to nt xxx-xxx) and GCGGGCAGAGGATCCTGGGGCTGTCTGGCCTCACAGCACT [SEQ ID NO:14] were used. The probes were labeled at the 3′-end with deoxyadenosine-alpha[thio]triphosphate [³⁵S] (NEN, USA) using terminal deoxynucleotidyl transferase (IBI, USA) to a specific activity of 7-10×10⁸ cpm/μg and hybridised to the sections without pretreatment for 16-18 h at 42° C. The hybridisation mixture contained: 50% v/v formamide, 4×SSC (1×SSC=0.15M NaCl and 0.015M sodium-citrate), 1× Denhardt's solution (0.02% each of polyvinyl-pyrrolidone, BSA and Ficoll), 1% v/v sarcosyl (N-lauroylsarcosine; Sigma), 0.02M phosphate buffer (pH 7.0), 10% w/v dextran sulfate (Pharmacia, Sweden), 250 μg/ml yeast tRNA (Sigma), 500 μg/ml sheared and heat denatured salmon sperm DNA (Sigma) and 200 mM dithiothreitol (DTT; LKB, Sweden). In control sections, the specificity of both probes was checked by adding a 20-fold excess of unlabeled probe to the hybridisation mixture. In addition, adjacent sections were hybridised with a probe unrelated to this study which gave a different expression pattern. Following hybridisation the sections were washed several times in 1×SSC at 55° C., dehydrated in ethanol and dipped in NTB2 nuclear track emulsion (Kodak, USA). After 3-5 weeks the sections were development in D-19 developer (Kodak, USA) and cover-slipped. In some cases, sections were opposed to an autoradiographic film (Beta-max autoradiography film Amersham Ltd, UK) prior to emulsion-dipping.

The four different probes gave identical hybridisation patterns in all tissues examined. Mouse VRF expression was detecting already in the E8 embryo, in which positive signal was recorded over structures most likely corresponding to the neuronal tube. In sagittal sections of E14 mouse embryo the strongest hybridisation signal was present over heart and in the nervous system, especially cerebral cortex (FIG. 14A). A low level of expression was present in all other tissues. At a later gestational age, E17, a high mVRF mRNA signal was confined to the heart and brown fat tissue in the back and around the neck (FIG. 14B). Clearly positive hybridisation signals were present in the gray of the spinal cord and in the tongue (FIG. 14B). Expression in the cerebral cortex was clearly reduced compared to day 14. The weak background expression seen in the E14 embryo in for example muscle, had decreased at this gestational age. A strong mVRF mRNA hybridisation signal was present solely over the heart and in the brown fat in the young adult mice (FIG. 14C). The signal over the heart was evenly distributed over the entire ventricular wall, including the papillary muscles (FIG. 14D). In sections of heart tissue hybridised with an excess of cold probe, no specific labeling over background signal was recorded (FIG. 14E).

Apart from the heart, mVRF mRNA signal was present over certain tissues on the outside of the thoracic cage that morphologically resembled brown fat. This was verified with sudan black counterstaining, which showed a strong staining in the same areas (FIGS. 15A and 15B). In transverse sections of adult mouse spinal cord, the mVRF probes gave a neuronal staining pattern over the gray matter (FIG. 15C). Counterstaining with toluidine (FIG. 15D) showed that motoneurons in the ventral horn (FIGS. 15C and 15D), interneurons (FIG. 15C) in the deep part of the dorsal horn and around the central canal where to a large extent positive for mVRF mRNA.

### EXAMPLE 7

### Effects of VEGF and SOM175 Proteins on Chick Sensory Neurons

The effects of VEGF and SOM175 proteins on embryonic day 8 chick sensory neurons were determined using the method of Nurcombe et al (1992). The neuronal assay was read at 48 hours using 2000 cells per assay well. The results were obtained using ³H-thymidine counts. The percentage survival of neurons, neurite outgrowth and average neurite length in μm were determined using NGF as positive control and various concentrations of VEGF, VEGF in the presence of heparin and VEGF in the presence of heparin and 5 μM, 5′-flurouracil (5FU). 5FU kills glial cells.

The results are shown in FIG. 16. The results show that VEGF is effective in promoting neuronal survival but that this requires the presence of glial cells. FIG. 17 shows the results of the effect of VEGF and SOM175 on three types of chick glia. The glia tested were CNS glia, peripheral glia and CNS oligodendrocytes. Heparin was used as 10 μg/ml in all cultures and the assay was read at 24 hours. Results were measured in ³H-thymidine counts using 2000 cells per well.

The results show that for chick central and peripheral neurons, astroglia were markedly stimulated to proliferate by SOM175 in the presence of heparin but that chick oligodendrocytes showed negligible increase in the rate of division.

### EXAMPLE 8

### Effects OF SOM175 Proteins on Mouse Primary and Central Neurons

The results in Example 7 show that VEGF isoform had an effect on chick primary and central neurons through the agency of the astroglial cells. Similar experiments were repeated in mouse cells.

Culture Conditions

Neuronal and gligal cells for all in vitro experiments were prepared and cultured according to the techniques described in “Methods in Neurosciences (Vol. 2): Cell Culture” Ed. P. M. Conn, Academic Press, San Diego, 1990, pp 33-46 for astroglial cells, pp 56-74 for oligodendroglial cells, and pp 87-102 for central neurons.

Cells were plated onto 24-well culture clusters (Nunc) coated with poly-L-ornithine (0.1 mg/ml, 1 h) at a density of 2,000 cells/well. After 48 hours in culture, neurons were counted in the wells under inverted phase light using well established techniques (Maruta et al. 1993) and glial cells assessed with [³H]thymidine uptake to monitor cell division rates as below. Heparin (10 μg/ml, low molecular weight fraction, Sigma Chemical Corp.) was present at all times in the culture media except where noted. The neuronal cultures were supplemented with 5 mM 5-fluoro-2-deoxyuridine (Sigma) to suppress background glial growth.

³H-Thymidine Incorporation Assay for Glial Cell Proliferation

The cells were pulsed for 14 h with ³H-thymidine (specific activity 103 μCi/ug) from a stock concentration of 0.1 mCi/ml in standard medium, giving a final incubating volume of 20 μl/well. The contents of the wells were harvested and absorbed onto nitrocellulose paper (Titertek, Flow). Remaining adherent cells were removed by incubation with trypsin/versene (CSL Limited, Victoria, Australia) for 5 min. This procedure was carried out twice. The nitrocellulose discs were washed in a standard Titertek harvester (Flow) using first distilled water, and then methanol. The nitrocellulose discs were dried, scintillation fluid (containing 5% v/v Triton-X) added and the discs counted on a scintillation counter.

Greatest activity was seen with preparations of SOM175 absent exon 6 (SOMΔX6) on mouse astroglial cell cultures, where there was a significant stimulus to their proliferation when delivered in conjunction with heparin (FIG. 16). Little stimulus was given to the proliferation of oligodendroglial cells (FIG. 17), and very little discernable potentiation of the survival response of isolated forebrain neurons (FIG. 18). The standard deviation on all three graphs for each point was less than 8%.

The viability of neurons can be maintained by promoting glial cell proliferation. Furthermore, SOMΔX6 is a good inducer of astroglial proliferation and may be expressed in conjunction with the formation of astroglial endfeet on central nervous system endothelial cells.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 3
Splice junctions of the murine VRF gene

| | | | | | |
|---|---|---|---|---|---|
| 5′ UTR* . . . | Exon 1 | >223 bp | CCCAGgtacgtgcgt | Intron I | 495 bp |
| ttccccacagGCCCC | Exon 2 | 43 bp | GAAAGgtaataatag | Intron II | 288 bp |
| ctgcccacagTGGTG | Exon 3 | 197 bp | TGCAGgtaccagggc | Intron III | 196 bp |
| ctgagcacagATCCT | Exon 4 | 74 bp | TGCAGgtgccagccc | Intron IV | 182 bp |
| ctcttttcagACCTA | Exon 5 | 36 bp | GACAGattcttggtg | Intron V | 191 bp |
| ctcctcctagGGTTG | Exon 6 | 101 bp | | (no intron) | |
| CCCACTCCAGCCCCA | Exon 7 | 135 bp | TGTAGgtaaggagtc | Intron VI | ˜2200 bp |
| cactccccagGTGCC | Exon 8 | 394 bp | AGAGATGGAGACACT | | |

Uppercase and lowercase letters denote exonic and intronic sequences respectively.
*Indicates that the 5′ end of exon 1 has not yet been determined.

### BIBLIOGRAPHY

Adams M D, Soares M B, Kerlavage A R, Fields C, Venter J C, (1993) Nature Genet, 4, 373-380.
Birnstiel M L, Busslinger M and Strub K (1985) Cell 41, 349-359.
Breier G, Albrecht U, Sterrer S and Risau W (1992) Development 114, 521-532.
Chomczynski P and Sacchi N (1987) Analyt. Biochem. 162, 156-159.
Church G and Gilbert W (1984) Proc. Natl. Acad. Sci. USA 18, 1991-1995.
Dagerlind A, Friberg K, Bean A J and Hokfelt T (1992) Histochemistry 98, 39-49.
Dissen G A, Lara H E, Fabrenbach W H, Costa M E, Ojeda S R, (1994) Endocrinology 134, 1146-1154.
Drinkwater C C, Barker P A, Suter U and Shooter E M (1993) J. Biol. Chem., 268, 23202-23207.
Drinkwater C C, Suter U, Angst C and Shooter E M (1991) Proc. Roy. Soc. Lond (Series B), 246, 307-313.
Ewton D Z & Florini J R (1980) Endocrinology, 106: 577-583.
Ferrara N & Henzel W J (1989) Biochem. Biophys. Res. Commun. 161, 851-858.
Folkman J & Shing Y (1992) J. Biol. Chem. 267, 10931-10934.
Gospodarowicz D, Abraham J A & Schilling J (1989) Proc. Natl. Acad Sci USA 86, 7311-7315.
Gospodarowicz D, Weseman J, Morgan J S & Lindstrom J (1976) J. Cell Biol., 70: 395-405.
Hagg T, Quon D, Higaki J & Varon S (1992) Neuron, 8, 145-158.
Hefti S (1986) J. Neurosci, 6, 2155-2162.
Hendry I A & Campbell J (1976) J. Neurocytol., 5, 351-360.
Hendry I A, Murphy M, Hilton D J, Nicola N A & Bartlett P F (1992) J. Neurosci. 12, 3427-3434.
Hunt et al., (1967) Am. J. Surgery, 114: 302-307.
Koch A E, Harlow L A, Haines G K, Amento E P, Unemoti E N, Wong W L, Pope R M, Ferrara N, (1994) J. Immunol. 152, 4149-4156.
Kromer A F (1987) Science, 235, 214-216.
Larsson C, Weber G, Kvanta E, Lewis C, Janson M, Jones C, Glaser T, Evans G, Nordenskjold M, (1992) Hum. Genet. 89, 187-193.
OZUS Leung D W, Cachianes G, Kuang W-J, Goeddel D V & Ferrara N (1989) Science 246:1306-1309.
Lowe C, Cornish J, Callon K, Martin T J & Reid I R (1991) J. Bone Mineral Res., 6, 1277-1283.
Lowe C, Cornish J, Martin T J & Reid I R (1991) Calcif. Tissue Int., 49, 394-397.
Martinou J C, Martinou I & Kato A C (1992) Neuron, 8, 737-744.
Maruta et al (1993) Growth Factors 8: 119-134.
Midy V & Plouet J (1994) Biochem. Biophys. Res. Commun., 199: 380-386.
Miles A A & Miles E M (1952) J. Physiol. (Lond) 118:228-257.
Montesano R, Vassalli J D, Baird A, Guillemin R & Orci, L (1986) Proc. Natl. Acad. Sci. USA, 83, 7297-7301.
Nurcombe et al (1992) Development 116: 1175-1183.
Otto D., Frotscher M & Unsicker K (1989) J. Neurosci. Res., 22, 83-91.
Pepper M S, Ferrara N, Orci L, Montesano R. (1991) Biochem. Biophys. Res. Commun. 181, 902-906).
Rochell J M, Watson M L, Oakey R J and Seldin M F (1992) Genomics 14, 26-31.
Roth S & Weston J (1967) Proc. Natl. Acad Sci USA, 58: 974-980.
Sambrook J, Fritsch E F, Maniatis T, (1989) Molecular Cloning: A Laboratory Manual-2nd Ed. Cold Spring Harbor Laboratory Press, Cold Spring Harbor, N.Y.
Santos M A (1991) Nucleic Acids Res. 19, 5442.
Schilling et al., (1959) Surgery, 46: 702-710.
Senger D R, Van De Water L, Brown L F, Nagy J A, Yeo K T, Yeo T K, Berse B, Jackman R W, Dvorak A M, Dvorak H F (1993) Cancer Netastasis Rev. 12, 303-324.
Sharkey A M, Chamock-Jones D S, Boocock C A, Brown K D, Smith S K, (1993) J. Reprod Fertil. 99, 609-615.
Sunderkotter C, Steinbrink K, Goebeler M, Bhardway R, Sorg E, (1993) J. Leukocyt, Biol. 55, 410-422.
Suter U, Angst C, Tien C-L, Drinkwater C C, Lindsay R M and Shooter E M (1992) J. Neurosci., 12, 306-318.
Tischer E, Mitchell R, Hartman T, Silva M, Gospodarowicz D, Fiddes J C, & Abraham J (1991) J. Biol. Chem. 266, 11947-11954.
Williams L R, Varon S, Peterson G M, Wictorin K, Fischer W, Bjorklund A & Gage F H (1986) Proc. Natl. Acad. Sci. USA 83, 9231-9235.
Yan Z, Weich H A, Bernart W, Breckwoldt M, Neulen J, (1993) J. Clin. Endocrinol. Metab. 77, 1723-1725.
Yip N K, Rich K M, Lampe P A & Johnson E M Jr (1984) J. Neurosci., 4, 2986-2992.

1. An isolated polypeptide comprising the amino acid sequence of SEQ ID NO: 4.

2. The isolated polypeptide of claim 1 consisting of the amino acid sequence of SEQ ID NO: 4.

3. The isolated polypeptide comprising the amino acid sequence of SEQ ID NO: 17.

4. The isolated polypeptide of claim 3 consisting of the amino acid sequence of SEQ ID NO: 17.

5. A composition comprising the polypeptide of claim 1.

6. A composition comprising the polypeptide of claim 3.

7. An isolated nucleic acid encoding a polypeptide comprising the amino acid sequence of SEQ ID NO: 4.

8. The isolated nucleic acid of claim 7 comprising the nucleotide sequence of SEQ ID NO: 3.

9. An isolated nucleic acid encoding a polypeptide comprising the amino acid sequence of SEQ ID NO: 17.

10. The isolated nucleic acid of claim 9 comprising the nucleotide sequence of SEQ ID NO: 16.

11. An antibody to a polypeptide comprising the amino sequence of SEQ ID NO: 4.

12. An antibody to a polypeptide comprising the amino acid sequence of SEQ ID NO: 17.
